SEQUENCE LISTING (22 sequences)

SEQ ID NO: 1 - DNA, 15 bp, synthetic construct
  source 1..15: mol_type = other DNA
ggttggtgtggttgg

SEQ ID NO: 2 - DNA, 29 bp, synthetic construct
  source 1..29: mol_type = other DNA
agtccgtggtagggcaggttggggtgact

SEQ ID NO: 3-10 - skipped (8 entries)

SEQ ID NO: 11 - DNA, 10 bp, synthetic construct
  source 1..10: mol_type = other DNA
gtcgtaagct

SEQ ID NO: 12 - DNA, 10 bp, synthetic construct
  source 1..10: mol_type = other DNA
agcttacgac

SEQ ID NO: 13 - DNA, 21 bp, synthetic construct
  source 1..21: mol_type = other DNA
  misc_feature 15^16: note = (PEG)6 linker
ggttggtgtggttgggtcgta

SEQ ID NO: 14 - DNA, 35 bp, synthetic construct
  source 1..35: mol_type = other DNA
  misc_feature 6^7: note = (PEG)6 linker
tacgacagtccgtggtagggcaggttggggtgact

SEQ ID NO: 15 - DNA, 22 bp, synthetic construct
  source 1..22: mol_type = other DNA
  misc_feature 15^16: note = (PEG)6 linker
ggttggtgtggttgggtcgtat

SEQ ID NO: 16 - DNA, 36 bp, synthetic construct
  source 1..36: mol_type = other DNA
  misc_feature 7^8: note = (PEG)6 linker
atacgacagtccgtggtagggcaggttggggtgact

SEQ ID NO: 17 - DNA, 23 bp, synthetic construct
  source 1..23: mol_type = other DNA
  misc_feature 15^16: note = (PEG)6 linker
ggttggtgtggttgggtcgtagt

SEQ ID NO: 18 - DNA, 37 bp, synthetic construct
  source 1..37: mol_type = other DNA
  misc_feature 8^9: note = (PEG)6 linker
actacgacagtccgtggtagggcaggttggggtgact

SEQ ID NO: 19 - DNA, 24 bp, synthetic construct
  source 1..24: mol_type = other DNA
  misc_feature 15^16: note = (PEG)6 linker
ggttggtgtggttgggtcgtaagt

SEQ ID NO: 20 - DNA, 38 bp, synthetic construct
  source 1..38: mol_type = other DNA
  misc_feature 9^10: note = (PEG)6 linker
acttacgacagtccgtggtagggcaggttggggtgact

SEQ ID NO: 21 - DNA, 25 bp, synthetic construct
  source 1..25: mol_type = other DNA
  misc_feature 15^16: note = (PEG)6 linker
ggttggtgtggttgggtcgtaagct

SEQ ID NO: 22 - DNA, 39 bp, synthetic construct
  source 1..39: mol_type = other DNA
  misc_feature 10^11: note = (PEG)6 linker
agcttacgacagtccgtggtagggcaggttggggtgact

(19) United States
(12) Patent Application Publication
(10) Pub. No.: US 2025/0076264 A1
(43) Pub. Date: Mar. 6, 2025

(54) POINT OF CARE TESTING SYSTEM FOR ANTITHROMBIN III

(71) Applicant: University of Florida Research Foundation, Incorporated, Gainesville, FL (US)

(72) Inventors: Adam S. Veige, Gainesville, FL (US); Yong Zeng, Gainesville, FL (US); Bruce Davis Spiess, Gainesville, FL (US); Amanda Orta Santana, Gainesville, FL (US); He Yan, Gainesville, FL (US)

(21) Appl. No.: 18/726,489

(22) PCT Filed: Jan. 23, 2023
(86) PCT No.: PCT/US2023/061059

§ 371 Date: Jul. 3, 2024

Related U.S. Application Data
(60) Provisional application No. 63/302,395, filed on Jan. 24, 2022.
(60) Provisional application No. 63/368,212, filed on Jul. 12, 2022.

(51) Int. Cl. G01N 30/88; G01N 33/542; G01N 33/58; G01N 33/68

(52) U.S. Cl. CPC G01N 30/88; G01N 33/542; G01N 33/582; G01N 33/6839; G01N 2030/8831; G01N 2333/8128

(57) ABSTRACT

Compounds and compositions for determining the level of antithrombin (ATIII) in a sample are described along with a system for determining the level of ATIII in a point-of-care setting. Methods of forming the compounds and compositions are also described. Methods of using the compounds and compositions to quantify the level of ATIII in a subject are further described. A system and apparatus are provided that determine the level of ATIII in a point-of-care setting in an efficient manner to facilitate determining a dosage or heparin or ATIII to administer to a patient.

### CROSS-REFERENCE TO RELATED APPLICATIONS

This application claims the benefit of U.S. Provisional Application No. 63/302,395 filed Jan. 24, 2022, and U.S. Provisional Application No. 63/368,212, filed Jul. 12, 2022, each of which is incorporated herein by reference.

### SEQUENCE LISTING

The Sequence Listing written in file 573852_SeqListing_ST26.xml is 19 kilobytes in size, was created Dec. 1, 2022, and is hereby incorporated by reference.

### TECHNOLOGICAL FIELD

Embodiments of the present disclosure relate generally to testing the level of Antithrombin III in a patient, and more particularly, to a point-of-care testing system for testing the level of Antithrombin III in a patient in a care setting with a result obtained quickly and efficiently.

### BACKGROUND

Anti-thrombin (ATIII) is a circulating serpin (serine protease inhibitor) protein produced by the liver that functions as a modulator/buffer of inflammation and coagulation. ATIII inhibits inflammation and coagulation by binding to and inhibiting thrombin. If humans are born with <20% ATIII activity they exhibit severe hypercoagulability and most die within the first few days of birth. Levels between 30-60% are associated with chronic and sometime acute life-threatening conditions. ATIII deficiency is one of the most common causes of deep venous thrombosis and spontaneous pulmonary embolism.

Heparin is the most commonly utilized intravenous agent for anticoagulation in hospitals today. Heparin acts as an anticoagulant through its interaction with ATIII. Anti-thrombin activity of ATIII is increased 2000-4000 fold by binding the heparin. Approximately 15% of all patients entering a hospital receive heparin at some time during their visit. It is required for heart-lung machine use. In heart and vascular surgery, heparin is given in moderate to large dosages (250-600 u/Kg of heparin). Smaller dosages are utilized routinely for procedures such as cardiac catheterization, renal dialysis and many other blood filtration or radiology procedures.

Patient response to heparin is highly variable because of polymorphisms in the population and because various diseases, injuries, conditions, or medications can decrease circulating ATIII. In particular, heparin treatment results in decreased circulating ATIII. In heart surgery, the variability of human response to heparin is a major problem. In patients with low ATIII, heparin fails to function adequately as an anticoagulant. Such patients require administration of exogenous ATIII. Antithrombin supplementation results in better survival in cardiac surgery patients. However, excess antithrombin can lead to deleterious bleeding events. Ideally, the level of ATIII in a subject would be determined prior to administration of heparin or ATIII. Currently there are no reliable means to predict, or quickly measure ATIII levels in a subject. Existing tests for ATIII indirectly assess anti-Xa activity in the presence or absence of heparin or utilize enzyme or immuno assays that a complex and/or infrequently run in hospital setting.

### SUMMARY

Described are compounds, compositions, and kits useful for determining the level of anti-thrombin III (ATIII) in a sample. The compounds, compositions, and kits comprise signaling aptamers that bind to thrombin and produce a detectable signal in the presence of ATIII. In some embodiments, the signaling aptamers comprise a first thrombin-specific aptamer containing a fluorescent label and a second thrombin-specific aptamer containing a quencher. In some embodiments, the signaling aptamers are provided bound to thrombin in a thrombin-signaling aptamer complex. When bound to thrombin, hybridizing sequences present on each of the signaling aptamers hybridize to form a duplex region. Formation of the duplex places the fluorescent label in proximity to the quencher, resulting in decreased fluorescence of the fluorescent label. In the presence to ATIII, the ATIII binds to the thrombin and displaces the first signaling aptamer, the second signaling aptamer, or both signaling aptamers. The displacing of the signaling the aptamer(s) results in an increase in fluorescence signal. The increase in fluorescence signal can be measured and used to detect and/or quantify ATIII in a sample.

In some embodiments, the kits further contain heparin. Proteins present in a sample, such as a blood sample, can disrupt the aptamer-thrombin complex. ATIII-independent disruption of the aptamer-thrombin complex can lead to overestimation of the amount of ATIII in the sample. Heparin can be added to the assay to selectively activate ATIII towards the aptamer complex. Heparin enhances the activity of ATIII and accelerates ATIII binding of thrombin. Signal output is higher at earlier times with ATIII is activated with heparin than when no heparin is present. Any change in the initial rate of signal production upon adding heparin, is attributed to the heparin-activated ATIII dependent signal, and not from background protein interactions. Thus, the change in initial rate of fluorescent increase in the assay, when comparing samples tested in the presence of heparin with sample tested in the absence of heparin can be correlated to ATIII concentration in the blood.

In some embodiments, heparin is attached to a solid support and used to extract ATIII from the sample prior to contact with the thrombin-signaling aptamer complex.

Methods of using the described compounds, compositions, and kits to detect and/or quantify the amount of ATIII in a sample are also described. In some embodiments, the methods comprise: forming a thrombin-signaling aptamer complex comprising thrombin, a first signaling aptamer, and a second signaling aptamer, contacting the thrombin-signaling aptamer complex with a sample containing or suspected of containing ATIII, and measuring fluorescence emitted by the fluorescent label. In some embodiments, the methods comprise contacting a thrombin-signaling aptamer complex with a sample containing or suspected of containing ATIII and detecting or measuring an increase in fluorescence signal emitted by the fluorescent label. In some embodiments, the increase in fluorescence signal is proportional to the level of ATIII in the sample.

In some embodiments, the methods comprise: forming a thrombin-signaling aptamer complex comprising thrombin, a first signaling aptamer, and a second signaling aptamer, contacting the thrombin-signaling aptamer complex with a sample containing or suspected of containing ATIII, and measuring initial rate fluorescence increase emitted by the fluorescent label in the presence and absence of heparin. In some embodiments, the methods comprise contacting a thrombin-signaling aptamer complex with a sample containing or suspected of containing ATIII and detecting or measuring an initial rate of increase in fluorescence signal emitted by the fluorescent label in the presence and absence of heparin. In some embodiments, the change in initial rate fluorescence signal increase in the sample containing heparin compared to the initial rate of fluorescence signal increase in the sample lacking heparin is proportional to the level of ATIII in the sample.

In some embodiments, the methods comprise: contacting sample containing or suspected of containing ATIII with heparin linked to a solid support, purifying any ATII in the sample from one or more other components of the sample using the heparin linked to the solid support, forming a thrombin-signaling aptamer complex comprising thrombin, a first signaling aptamer, and a second signaling aptamer, contacting the thrombin-signaling aptamer complex with the purified sample, and measuring fluorescence emitted by the fluorescent label. In some embodiments, the methods comprise contacting sample containing or suspected of containing ATIII with heparin linked to a solid support, purifying any ATII in the sample from one or more other components of the sample using the heparin linked to the solid support, contacting a thrombin-signaling aptamer complex with a purified sample containing or suspected of containing ATIII, and measuring fluorescence signal emitted by the fluorescent label. In some embodiments, the increase in fluorescence signal is proportional to the level of ATIII in the sample.

In some embodiments, the described compounds, compositions, kits, and methods can be used to determine the dosage of heparin and/or exogenous ATIII to administer to a subject prior to, concurrent with, or subsequent to a medical procedure.

Embodiments provided herein include a system for determining a level of anti-thrombin (ATIII) in a subject, the system including: a column defining an inlet and an outlet, where heparin beads are retained within the column; a manifold connected to the inlet of the column, where the manifold includes a plurality of ports, each port configured to receive a fluid, where the manifold conducts the fluid from each port to the inlet of the column; a first syringe including a washing buffer received at a first port of the plurality of ports; a second syringe including plasma received at a second port of the plurality of ports of the manifold; a third syringe including a washing buffer received at a third port of the plurality of ports of the manifold; and a fourth syringe including an aptamer-thrombin complex received at a fourth port of the plurality of ports of the manifold, wherein the aptamer-thrombin complex comprises a first signaling aptamer comprising a first thrombin-specific aptamer, a first hybridization sequence, and a fluorescent label, a second signaling aptamer comprising a second thrombin-specific aptamer, a second hybridization sequence, and a quencher and thrombin, wherein the first and second hybridization sequences are complementary to each other, and wherein fluorescence of the fluorescent label is quenched when the first and second thrombin-specific signaling aptamers are bound to the thrombin.

According to some embodiments, the system includes a substrate, where the substrate includes a waste reservoir separated from the column by a waste valve and a fluorescence reservoir separated from the column by a fluorescence valve. The heparin beads retained within the column are, in some embodiments, washed in response to the first syringe driving the washing buffer through the inlet of the column. The heparin beads of some embodiments are incubated with the plasma in response to the second syringe driving the plasma through the inlet of the column, where the heparin beads capture thereon ATIII during incubation with the plasma. The heparin beads with the captured ATII are, in some embodiments, washed by the washing buffer in response to the third syringe driving the washing buffer through the inlet of the column. The heparin beads with the captured ATIII are, in some embodiments, incubated with the aptamer-thrombin complex in response to the fourth syringe driving the aptamer-thrombin complex through the inlet of the column, where the ATIII captured on the heparin beads trigger release of aptamer-F to a solution in the column for signal generation. According to some embodiments, fluorescence of the solution is read to determine a level of ATIII in the plasma.

Embodiments provided herein include a method for determining a level of anti-thrombin (ATIII) in a subject, the method including: driving a first washing buffer through a column within which are suspended heparin beads; driving plasma through the column; incubating the plasma with the heparin beads; driving a second washing buffer through the column; driving an aptamer-thrombin complex through the column; incubating the aptamer-thrombin complex with the heparin beads; and producing a fluorescence indicative of a level of ATIII within the plasma.

According to some embodiments, the first washing buffer is driven through the column by a first syringe, where the plasma is driven through the column by a second syringe, where the second washing buffer is driven through the column by a third syringe, and where the aptamer-thrombin complex is driven through the column by a fourth syringe. The method of some embodiments includes washing the heparin beads with the first washing buffer in response to driving the first washing buffer through the column within which the heparin beads are suspended. Methods of some embodiments include capturing the ATIII on the heparin beads in response to incubating the plasma with the heparin beads. The method of some embodiments includes washing the heparin beads with the captured ATIII with the second washing buffer in response to driving the second washing buffer through the column. Methods optionally include triggering release of aptamer-F to a solution in the column for signal generation in response to incubating the heparin beads with the captured ATIII with the aptamer-thrombin complex.

Embodiments provided herein include an apparatus for determining a level of anti-thrombin (ATIII) in a subject including: a column defining an inlet and an outlet; a manifold defining a plurality of ports and a manifold outlet, where the manifold outlet is connected to the inlet of the column; a substrate defining a waste flow path extending between a substrate inlet and a waste reservoir, and a fluorescence flow path extending between the substrate inlet and a fluorescence reservoir; a waste valve configured to open and close the waste flow path between the substrate inlet and the waste reservoir; and a fluorescence valve configured to open and close the fluorescence flow path between the substrate inlet and the fluorescence reservoir. The apparatus of some embodiments further includes: a first syringe received at a first port of the plurality of ports of the manifold; a second syringe received at a second port of the plurality of ports of the manifold; a third syringe received at a third port of the plurality of ports of the manifold; and a fourth syringe received at a fourth port of the plurality of ports of the manifold. According to certain embodiments, the first syringe holds a first washing buffer, the second syringe holds plasma, the third syringe holds a second washing buffer, and the fourth syringe holds an aptamer-thrombin complex. According to some embodiments, in response to sequential driving of contents of the first syringe, the second syringe, the third syringe, and the fourth syringe through the column, the fluorescence reservoir fluoresces based on a level of ATIII within the plasma.

### DESCRIPTION OF THE DRAWINGS

FIG. 1 illustrates a system for determining a level of anti-thrombin (ATIII) in a subject in a point-of-care setting according to an example embodiment of the present disclosure.

FIG. 2 illustrates the column suspending the heparin beads in a system for determining a level of ATIII in a subject in a point-of-care setting according to an example embodiment of the present disclosure.

FIG. 3 illustrates a workflow of the system for determining a level of anti-thrombin (ATIII) in a subject in a point-of-care setting according to an example embodiment of the present disclosure.

FIG. 4 is an illustration of ATIII (anti-thrombin) binding to a thrombin-signaling aptamer complex and displacing both the first (TBA15) and second (TBA29) signaling aptamers. For some signaling aptamer pairs, ATIII binding to thrombin results in dissociation of the first (e.g., TBA15) signaling aptamer.

FIG. 5 is a graph illustrating a fluorescence increase with increasing concentration of ATIII.

FIG. 6 is a graph illustrating fluorescence intensity at varying wavelengths for 100 nM signaling aptamer concentration. Complexes in the figure legend are in order of maximum intensity (highest to lowest maximum intensity).

FIG. 7 is a graph illustrating fluorescence intensity at varying wavelengths for 200 nM signaling aptamer concentration. Complexes in the figure legend are in order of maximum intensity (highest to lowest maximum intensity).

FIG. 8 is a graph illustrating fluorescence intensity at varying wavelengths for 300 nM signaling aptamer concentration. Complexes in the figure legend are in order of maximum intensity (highest to lowest maximum intensity).

FIG. 9 is a graph illustrating fluorescence intensity at varying wavelengths for 400 nM signaling aptamer concentration. Complexes in the figure legend are in order of maximum intensity (highest to lowest maximum intensity).

FIG. 10 is a graph illustrating fluorescence intensity at varying wavelengths for increasing ratio of thrombin to signaling aptamers TBA15-F6 and TBA29-D6. TBA15-F6 and TBA29-D6 were used as a concentration of 200 nM. Thrombin was used at 20 nM to 100 nM. Complexes in the figure legend are in order of maximum intensity (highest to lowest maximum intensity).

FIG. 11 is a graph illustrating fluorescence intensity at varying wavelengths for increasing ratio of thrombin to signaling aptamers TBA15-F7 and TBA29-D7. TBA15-D7 and TBA29-D7 were used as a concentration of 200 nM. Thrombin was used at 75 nM to 300 nM. Complexes in the figure legend are in order of maximum intensity (highest to lowest maximum intensity).

FIG. 12 is a graph illustrating fluorescence intensity at varying wavelengths for increasing ratio of thrombin to signaling aptamers TBA15-F8 and TBA29-D8. TBA15-D8 and TBA29-D8 were used as a concentration of 200 nM. Thrombin was used at 75 nM to 300 nM. Complexes in the figure legend are in order of maximum intensity (highest to lowest maximum intensity).

FIG. 13 is a graph illustrating fluorescence intensity at varying wavelengths for increasing ratio of thrombin to signaling aptamers TBA15-F9 and TBA29-D9. TBA15-D9 and TBA29-D9 were used as a concentration of 200 nM. Thrombin was used at 75 nM to 300 nM. Complexes in the figure legend are in order of maximum intensity (highest to lowest maximum intensity).

FIG. 14 is a graph illustrating fluorescence intensity at varying wavelengths for increasing ratio of thrombin to signaling aptamers TBA15-F10 and TBA29-D10. TBA15-D10 and TBA29-D10 were used as a concentration of 200 nM. Thrombin was used at 75 nM to 300 nM. Complexes in the figure legend are in order of maximum intensity (highest to lowest maximum intensity).

FIG. 15 is a graph illustrating kinetics of formation of TBA15-F9+TBA29-D9+thrombin (1:1:1) complexes at 200 nM for each component.

FIG. 16 is a graph illustrating kinetics of dissociation of TBA15-F9+TBA29-D9+thrombin (1:1:1) complex in the presence of 1940 nM ATIII. The complex was present at a concentration of 200 nM.

FIG. 17 is a graph illustrating fluorescence titration of 200 nM complex with increasing concentration, 0.25 g/L to 2.00 g/L, of heparin. Complexes in the figure legend are in order of maximum intensity (highest to lowest maximum intensity).

FIG. 18 is a graph illustrating fluorescence titration of 200 nM complex with increasing concentration, 1 g/L to 4 g/L, of HBA. Complexes in the figure legend are in order of maximum intensity (highest to lowest maximum intensity).

FIG. 19 is a graph illustrating fluorescence titration of 200 nM thrombin-signaling aptamer complex with increasing concentration of Human Serum Albumin (HSA) and ATIII.

FIG. 20 is a graph illustrating fluorescence titration of 200 nM thrombin-signaling aptamer complex with increasing concentration of heparin and ATIII.

FIG. 21 is a graph illustrating effect of heparin on kinetics of signal generation in aptamer ATIII assay.

FIG. 22 is a graph illustrating rate of fluorescence signal increase for varying amounts of ATIII in the absence of added heparin.

FIG. 23 is a graph illustrating rate of fluorescence signal increase for varying amounts of ATIII in the presence of added heparin.

FIG. 24 is a schematic depiction of heparin bound sepharose beads extracting ATIII, where remaining proteins are removed and the aptamer-thrombin assay is added to generate a fluorescence signal indicative of the ATIII originally present in the plasma sample.

FIG. 25 depicts a graph illustrating detection of ATIII:
dP+B=deficient plasma+beads (5 μL plasma was diluted in 95 μL PBS);
dP+A+B=deficient plasma+spiked ATIII (2 μM)+beads (5 uL plasma was diluted in 95 μL ATIII solution (2 μM); and
dP is the detection of deficient plasma without beads, and NC is the negative control with no ATIII added into the normal assay

FIG. 26 depicts graphs illustrating sensitivity of the heparin bead ATT assay with the upper graph shows RLUs for varying concentrations of ATII.

FIG. 27 is a graph illustrating fluorescence of TBA15-F9 (F), TBA15-F9+TBA29-D9 (F+Q), TBA15-F9+TBA29-D9+Thrombin (F+Q+T), and TBA15-F9+TBA29-D9+Thrombin+ATIII (F+Q+T+AT-III) in various buffers. For each buffer, bars are in order of: F, F+Q, F+Q+T, F+Q+T+AT-III.

FIG. 28 is a graph illustrating kinetics of formation of TBA15-F9+TBA29-D9+Thrombin complexes.

FIG. 29 is a graph used in determining limit of detection of ATIII

### DETAILED DESCRIPTION

### A. Definitions

Before describing the present teachings in detail, it is to be understood that the disclosure is not limited to specific compositions or process steps, as such may vary. It should be noted that, as used in this specification and the appended claims, the singular form “a,” “an,” and “the” include plural references unless the context clearly dictates otherwise. Thus, for example, reference to “an oligomer” includes a plurality of oligomers and the like. The conjunction “or” is to be interpreted in the inclusive sense, i.e., as equivalent to “and/or,” unless the inclusive sense would be unreasonable in the context.

In general, the term “about” indicates insubstantial variation in a quantity of a component of a composition not having any significant effect on the activity or stability of the composition. When the specification discloses a specific value for a parameter, the specification should be understood as alternatively disclosing the parameter at “about” that value. All ranges are to be interpreted as encompassing the endpoints in the absence of express exclusions such as “not including the endpoints”; thus, for example, “within 10-15” includes the values 10 and 15. Also, the use of “comprise,” “comprises,” “comprising,” “contain,” “contains,” “containing,” “include,” “includes,” and “including” are not intended to be limiting. It is to be understood that both the foregoing general description and detailed description are exemplary and explanatory only and are not restrictive of the teachings. To the extent that any material incorporated by reference is inconsistent with the express content of this disclosure, the express content controls.

Unless specifically noted, embodiments in the specification that recite “comprising” various components are also contemplated as “consisting of” or “consisting essentially of” the recited components. Embodiments in the specification that recite “consisting essentially of” various components are also contemplated as “consisting of”. “Consisting essentially of” means that additional component(s), composition(s) or method step(s) that do not materially change the basic and novel characteristics of the compositions and methods described herein may be included in those compositions or methods.

“Aptamers” are short (often less than 40 nucleobases in length) single-stranded polynucleotide molecules that selectively bind to specific molecular targets, such as proteins or protein epitopes. Aptamers are readily produced by chemical syntheses. Aptamers can possess desirable storage properties. In some embodiments, the aptamer elicits little or no immunogenicity in therapeutic applications.

A “signaling aptamer” is an aptamer linked to a hybridizing sequence and a label. The hybridizing sequence can be linked to the 5′ or 3′ end of the aptamer. In some embodiments, the hybridizing sequence is linked to the aptamer via a linker. In some embodiments, the label is linked to a hybridizing sequence.

A “hybridizing sequence” is a short (generally 6-12 nucleotides in length) single stranded polynucleotide capable of base pairing with a complementary hybridizing sequence. In some embodiments, a first signaling aptamer is linked to a first hybridizing sequence that is complementary a second hybridizing sequence linked to a second signaling aptamer. The first and second hybridizing sequences can base pair (hybridize) to form a duplex region.

A “label” is a detectable molecule and/or a quencher. Detectable molecules include, but are not limited to, fluorescent labels. A fluorescent label (fluorophore) is a fluorescent chemical compound that can re-emit light upon light excitation. Fluorescent labels absorb light energy of a specific wavelength and re-emit light at a longer wavelength. A quencher is a compound that decreases the fluorescence intensity of a fluorescent label. A quencher can absorb excitation energy emitted from a fluorescent molecule and dissipate the energy as heat or as a longer wavelength light. When a fluorescent label and a quencher are in sufficiently close proximity, the fluorescent label's emission is suppressed.

A fluorescent label can be, but is not limited to: xanthenes, FITC, FAM™, TET™, CAL FLUOR™ (Orange or Red), ALEXA FLUOR™, QUASAR™, fluorescein, hexachlorofluorescein (HEX), rhodamine, Carboxy-X-Rhodamine (ROX), tetramethylrhodamine, IAEDANS, EDANS, coumarin, BODIPY FL, lucifer yellow, eosine, erythrosine, Texas Red, cyanines, or CY dyes (e.g., Cy3, Cy3.5, Cy5, Cy5.5).

A quencher can be, but is not limited to, DABCYL, BLACK HOLE QUENCHERs™ (BHQ™, e.g., Black Hole Quencher-0, Black Hole Quencher 1, Black Hole Quencher 2, Black Hole Quencher 3, Black Hole Quencher 650), or TAMRA™ compounds.

Exemplary fluorescent label/quencher pairs include, but are not limited to, fluorescein and dabcyl, fluorescein and black hole quencher, eosine and DABCYL, coumarin/DABCYL, CY5 and Black Hole Quencher 1, CY5 and Black Hole Quencher 2, CY3 and Black Hole Quencher 1, CY3 and Black Hole Quencher 2

A “linker” or linking group is a connection between two atoms that links one chemical group or segment of interest (e.g., an aptamer) to another chemical group or segment of interest (e.g., a hybridizing sequence) via one or more covalent bonds. In some embodiments, a linker increases the distance between the two atoms. In some embodiments, a linker is a flexible linker that adds flexibility to the linkage. Linkers include, but at not limited to, alkyl groups, alkenyl groups, alkynyl groups, aryl groups, aralkyl groups, aralkenyl groups, and aralkynyl groups (each of which can contain one or more heteroatoms), heterocycles, amino acids, nucleotides, saccharides, and polymeric groups. Polymeric groups include, but are not limited to, polyethylene glycol. In some embodiments, a linker comprises PEGₙ, wherein n is an integer from 1 to 50. In some embodiments, the linker comprises PEG₁, PEG₂, PEG₃, PEG₄, PEG₅, PEG₆, PEG₇, PEG₈, PEG₉, or PEG₁₀. The linker does not interfere with binding of the aptamer to its molecular target (e.g., thrombin). The linker can facilitate duplex formation by complementary hybridizing sequences when two aptamers containing complementary hybridization sequences are bound to a target molecule.

The “sample” includes any physiological fluid derived from a subject. Samples include, but are not limited to, blood, serum, plasma, and fractions thereof. A sample can be pretreated prior to use, such as preparing plasma from blood. A sample may be used directly as obtained from the subject or following a pretreatment to modify the character of the sample. Pretreatments include, but are not limited to, filtration, dilution, and addition of one or more reagents useful in preparing or analyzing the sample. Sample includes the original sample or an original sample that has received one or more pretreatments. A sample may be of any suitable size or volume. In some embodiments, the sample volume is less than or equal to 1 mL, less than or equal to 500 μL, less than or equal to 250 μL, less than or equal to 100 μL, less than or equal to 75 μL, less than or equal to 50 μL, less than or equal to 35 μL, less than or equal to 25 μL, or less than or equal to 20 μL.

“Thrombin” is a serine protease. In humans, thrombin is encoded by the F2 gene. Prothrombin (coagulation factor II) is proteolytically cleaved to form thrombin in the clotting process. Thrombin in turn acts as a seine protease that converts soluble fibrinogen into insoluble strands of fibrin, as well as catalyzing other coagulation-related reactions. Thrombin is inactivated by antithrombin III.

Heparin is a naturally occurring polysaccharide (glycosaminoglycan) that inhibits coagulation (i.e., acts as an anticoagulant). Natural heparin consists of molecular chains of varying lengths, or molecular weights. Heparin can be unfractionated heparin or low-molecular-weight heparin. LMWHs are defined as heparin salts having an average molecular weight of less than 8000 Da and for which at least 60% of all chains have a molecular weight less than 8000 Da.

“Heparin beads” comprise heparin bound to (e.g., conjugated to or immobilized on) a support or matrix. The support or matrix can be but is not limited to: sepharose, sepharose beads, agarose beads, a gel, acrylic beads, silica. Heparin beads have been widely used in affinity purification of various heparin-binding proteins or ligands, such as antithrombin III,

“Separating” or “purifying” means that one or more components of a sample are removed or separated from other sample components. Sample components include cellular fragments, proteins, carbohydrates, lipids, and nucleic acids. Purifying does not connote any degree of purification.

### B. Purpose

ATIII is a serine protease inhibitor that when activated by a heparinoid glycosaminoglycans at a specific pentasaccharide sequence on the protein (ATIII) changes conformation so that it has 100-1000 fold increased binding affinity for thrombin. Thrombin is a powerful inflammatory and coagulation amplifier, released from endothelial cells, platelets and leukocytes in response to a wide range of pro-inflammatory stimuli. ATIII is manufactured in the liver as non-constitutive protein. The liver, with normal liver function in healthy people, produces a steady and consistent production of ATIII. In disease states, or where certain drugs such as heparin have been given, the levels of ATIII decrease. ATIII is carried in the plasma but binds to the endothelial glycocalyx surface (to heparin). As such, the interaction between bound heparin and ATIII creates part of the endothelial brush boarder barrier to inflammation and thrombosis. ATIII is vital in maintaining normal blood vessel function. Abnormalities in ATIII levels such as non-production or consumption, or the presence of polymorphic less functional ATIII can lead to the formation of venous, and more rarely-arterial, clot formation with embolization. ATIII genetic dysfunctionality is perhaps the largest cause of spontaneous deep venous thrombosis (DVT) and pulmonary embolism (PE). ATIII not only binds to thrombin but also inhibits other coagulation cascade proteins in the contact activation cascade. Evidence suggests that cascade is a direct link from tissue destruction to bradykinin and compliment activation. Therefore ATIII is a buffer on that linkage creating a system where small amounts of tissue injury do not create whole body inflammatory events.

ATIII levels drop in the plasma during a number of disease states. In sepsis, ATIII levels drop and in severe sepsis levels are profoundly low. Trauma and vascular catastrophe such as aortic dissection cause immediate and rapid reductions in circulating ATIII. Pre-Eclampsia (toxicity of pregnancy) and Eclampsia see sudden catastrophic reductions in ATIII. In trauma and probably in toxicity of pregnancy ATIII reduction is due to the production or release of large amounts of tissue factor and thrombin. Liver disease causes reductions in the production of ATIII. But by far the largest cause of reduction of ATIII is the use of the drug heparin.

Heparin is an injectable complex of polysaccharides that contain the key pentasaccharide sequence to activate ATIII. High molecular weight (HMW) heparin is isolated from swine intestine and the commercially available drug is a mixture of active and inactive (>60% by weight) diffusely distributed molecular weight species of polysaccharides. Low molecular weight (LMW) heparins are more “refined”, more “targeted” and specific, but are less easily controlled with no active reversal agents. HMW heparin and now to some extent LMW heparin are the most widely utilized injectable drugs in the world in medicine. It is estimated that 30-60% of all patients entering a hospital for more than 24 hours are treated with heparin. Heparin consumes ATIII, the natural buffering capacity of the body for inflammation. Heparin also replaces or displaces the natural heparan form the endothelial system and makes the vascular tree therefore more prone to inflammatory attack. Heparin also is highly antigenic and leads to heparinPF4 antibodies in most humans exposed to the drug for prolonged periods of time. Heparin is utilized in surgery widely. Vascular surgery and open-heart surgery would not be possible today without the use of injectable large dose heparin and the reversal of that with another protein—protamine. At cardiac surgery the mean drop in ATIII levels is from normal 80-120% activity towards 40-60% activity. However, a significant outlier group of patients see profound potentially catastrophic drops in ATIII to levels in 15-30% of normal activity. These levels are similar to those found in non-survivable congenital ATIII deficiency or profound post trauma massive coagulopathy of trauma which leads to diffuse intravascular coagulation (DIC).

ATIII levels are able to be monitored in plasma by most hospital central laboratories. A chromogenic assay is performed on isolated, spun plasma and it is reported out as % of normal activity. Therefore, it is a functional assay of activity. Such a chromogenic assay is available in some hospitals on a STAT basis, but in most hospitals, ATIII testing is available on a 24 hour turn around standard laboratory testing time frame. As such, doctors do not routinely utilize ATIII testing to guide therapy. ATIII levels can and do change quickly, are predictive of potential coagulopathy, and are important in the most widely utilized drug in medicine today (heparin), yet it is rarely tested for as a protein level. The rapid changes and relatively long turnaround time limit the effectiveness of monitoring ATIII levels as they can change substantially before a result is returned.

Medical protocols have found ways to utilize heparin by employing surrogate tests for coagulation such as the Activated Partial Thromboplastin Time (APTT) and the Activated Coagulation Time (ACT). Both are dependent upon a number or protein (and cellular-ACT) interactions. The tests have some relationships to ATIII activity, but neither directly measures it, nor do they act in ways to enable ATIII titration. As a result, the monitoring of ATIII levels in standard medical practice and in disease states lacks precision, accuracy, and speed.

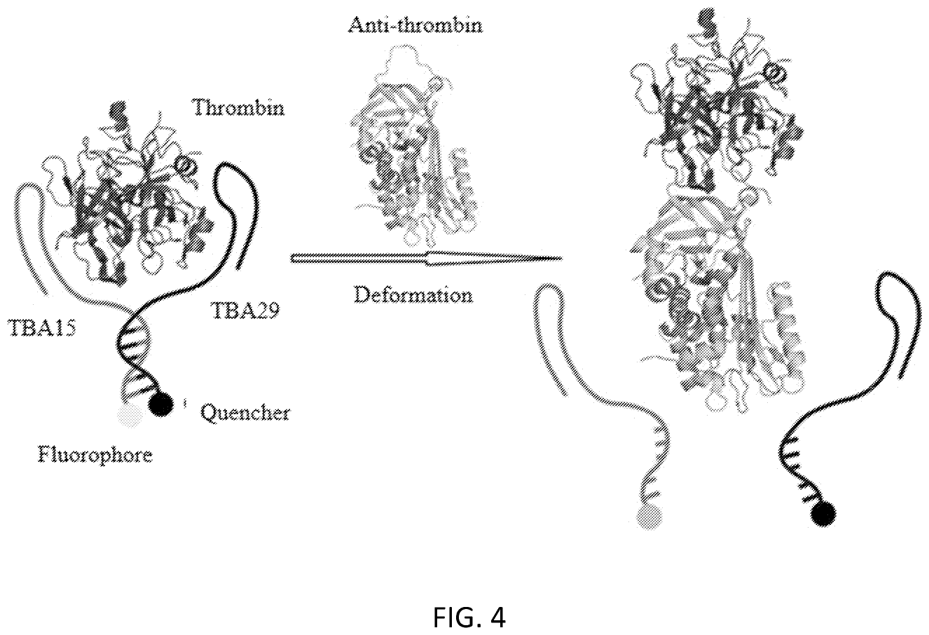

Embodiments provided herein provide a point-of-care testing system for testing the level of ATIII in a patient in a care setting with a result obtained relatively quickly as compared to traditional laboratory-based methods. Embodiments enable a real-time point-of-care test based on the development of a biochemical method for the detection of ATIII. Embodiments include a dual aptamer-based thrombin ATIII binding assay that is capable of quantitative detection of pure ATIII spiked in saline and human plasma. Embodiments use two complementary aptamers to bind to Thrombin and their fluorophores arrange in space to quench each other (on-state) as shown in FIG. 4. The intensity of the fluorescence is a direct correlation to the concentration of ATIII.

### C. Signaling Aptamers

Described are compounds, compositions, kits, and methods for detecting and/or quantifying the level of ATIII in a sample. Embodiments of the present disclosure provide a point-of-care testing system for measuring the level of ATIII in a patient in a care setting with a result obtained relatively quickly.

In some embodiments, the compounds and compositions comprise one or more signaling aptamers that bind to thrombin and produce a detectable signal in the presence of ATIII. In some embodiments, the compounds, compositions, and kits comprise a first signaling aptamer and a second signaling aptamer that each bind to thrombin. The first and second signaling aptamers bind to different epitopes on thrombin. In some embodiments, the first aptamer contains a detectable label, such as a fluorescent label, and the second aptamer contains a quencher that reduces or alters fluorescence of the fluorescent label. In some embodiments, the second aptamer contains a detectable label, such as a fluorescent label, and the first aptamer contains a quencher that reduces or alters fluorescence of the fluorescent label. The first and second signaling aptamers are configured such that the quencher quenches signal from the fluorescent label when both signaling aptamers are bound to thrombin. The first and second signaling aptamers are further designed such that ATIII binding to thrombin results in dissociation of at least one of the aptamers from the thrombin. Dissociation of the signaling aptamer from thrombin results in dequenching of the fluorescent label and an increase in detectable signal (i.e., fluorescence).

The first and second signaling aptamers contain hybridization sequences that are able to from a duplex region when the signaling aptamers are bound to thrombin. In some embodiments, the labels on the signaling aptamers are linked to the hybridization sequences. In some embodiments, the hybridization sequences are designed to position the fluorescent label in close proximity with the quencher when the hybridization sequences base pair to form a duplex. The hybridization sequences form a sufficiently stable duplex such that the fluorescent label is quenched when the signaling aptamers are bound to thrombin in the absence of ATIII. In some embodiments, the hybridization sequences are further designed such that binding of ATIII to thrombin results in dequenching and an increase in fluorescence.

In some embodiments, the signaling aptamers contain a linker between the aptamer sequence and the hybridization sequence. The linker can be used to increase spacing between the aptamer sequence and the hybridization sequence and/or to add flexibility between the aptamer sequence and the hybridization sequence. The linker is designed to facilitate duplex formation between hybridization sequences when the signaling aptamers are bound to thrombin.

In some embodiments, a first signaling aptamer contains the aptamer nucleotide sequence of thrombin aptamer TBA15. TBA15 is a 15-mer single stranded DNA having the sequence 5′-GGTTGGTGTGGTTGG-3′ (SEQ ID NO: 1). The TBA15 aptamer binds the exosite I epitope of thrombin with a Kd of about 100 nM. Exosite I is the binding site of fibrinogen on thrombin. In other embodiments, the first signaling aptamer contains the aptamer nucleotide sequence of thrombin aptamer TBA29 (described in more detail below). In some embodiments, the first signaling aptamer comprises a first hybridizing sequence linked to the 3′ end of the aptamer. In some embodiments, the first hybridizing sequence is linked to the 3′ end of the aptamer via a linker. In some embodiments, the linker comprises a PEG. In some embodiments, the PEG comprises PEG₁₋₁₀ (e.g., —(CH₂—CH₂—O)₁₋₁₀—). In some embodiments, the PEG comprises PEG₆. In some embodiments, the first signaling aptamer comprises a fluorescent molecule linked to the first hybridizing sequence. In some embodiments, the first signaling aptamer comprises a fluorescent molecule linked to the 3′ end of the first hybridizing sequence.

In some embodiments, a second signaling aptamer contains the aptamer nucleotide sequence of thrombin aptamer TBA29. TBA29 is a 29-mer single stranded DNA having the sequence 5′-AGTCCGTGGTAGGGCAGGTTGGGGTGACT-3′ (SEQ ID NO: 2). The TBA29 aptamer binds the exosite II epitope of thrombin with a Kd of about 0.5 nM. The exosite II epitope is involved in the activation of factor V/VIII and mediates the heparin binding. In other embodiments, the second signaling aptamer contains the aptamer nucleotide sequence of thrombin aptamer TBA15. In some embodiments, the second signaling aptamer comprises a second hybridizing sequence linked to the 5′ end of the aptamer. In some embodiments, the second hybridizing sequence is linked to the 5′ end of the aptamer via a linker. In some embodiments, the linker comprises a PEG. In some embodiments, the PEG comprises PEG₁₋₁₀ (e.g., —(CH₂—CH₂—O)₁₋₁₀—). In some embodiments, the PEG comprises PEG₆. In some embodiments, the second signaling aptamer comprises a quencher linked to the second hybridizing sequence. In some embodiments, the second signaling aptamer comprises a quencher linked to the 5′ end of the second hybridizing sequence.

Other thrombin-binding aptamers can be identified using methods known in the art. Such methods include, but are not limited to, systematic evolution of ligands by exponential enrichment (SELEX) and automated in vitro selection.

In some embodiments, the hybridizing sequences of the signaling aptamers are 5-20 nucleobases in length. In some embodiments, the hybridizing sequences of the signaling aptamers are 5, 6, 7, 8, 9, 10, 11, 12, 13, 14, 15, 16, 17, 18, 19, or 20 nucleobases in length. In some embodiments, the hybridizing sequences of the signaling aptamers are 6-10 nucleobases in length. In some embodiments, the hybridizing sequences of the signaling aptamers are 6, 7, 8, 9, or 10 nucleobases in length. In some embodiments, the hybridizing sequences of the signaling aptamers are 9 nucleobases in length.

In some embodiments, the hybridizing sequences of the signaling aptamers hybridize to form a duplex 6-10 base pairs in length. In some embodiments, the hybridizing sequences of the signaling aptamers hybridize to form a duplex 6, 7, 8, 9, or 10 base pairs in length. In some embodiments, the hybridizing sequence of the signaling aptamers hybridize to form a duplex 9 base pairs in length.

In some embodiments, a first signaling aptamer comprises a first hybridizing sequence of 5′-Nₙ-3′, wherein each N is independently any nucleobase and n is an integer from 6-10, and a second signaling aptamer comprises a second hybridizing sequence that is complementary to the first hybridizing sequence. In some embodiments, the hybridized duplex contains four G:C base pairs and 5 A:T base pairs.

In some embodiments, the hybridizing sequences of the signaling aptamers comprise or consist of the sequence 5′-GTCGTA-3′ (SEQ ID NO: 3) or 5′-TACGAC-3′ (SEQ ID NO: 4). In some embodiments, the first and second hybridizing sequences of the signaling aptamers comprise or consist of the sequences 5′-GTCGTA-3′ (SEQ ID NO: 3) and 5′-TACGAC-3′ (SEQ ID NO: 4).

In some embodiments, the hybridizing sequences of the signaling aptamers comprise or consist of the sequence 5′-GTCGTAT-3′ (SEQ ID NO: 5) or 5′-ATACGAC-3′ (SEQ ID NO: 6). In some embodiments, the first and second hybridizing sequences of the signaling aptamers comprise or consist of the sequences 5′-GTCGTAT-3′ (SEQ ID NO: 5) and 5′-ATACGAC-3′ (SEQ ID NO: 6).

In some embodiments, the hybridizing sequences of the signaling aptamers comprise or consist of the sequence 5′-GTCGTAGT-3′ (SEQ ID NO: 7) or 5′-ACTACGAC-3′ (SEQ ID NO: 8). In some embodiments, the first and second hybridizing sequences of the signaling aptamers comprise or consist of the sequences 5′-GTCGTAGT-3′ (SEQ ID NO: 7) and 5′-ACTACGAC-3′ (SEQ ID NO: 8).

In some embodiments, the hybridizing sequences of the signaling aptamers comprise or consist of the sequence 5′-GTCGTAAGT-3′ (SEQ ID NO: 9) or 5′-ACTTACGAC-3′ (SEQ ID NO: 10). In some embodiments, the first and second hybridizing sequences of the signaling aptamers comprise or consist of the sequences 5′-GTCGTAAGT-3′ (SEQ ID NO: 9) and 5′-ACTTACGAC-3′ (SEQ ID NO: 10).

In some embodiments, the hybridizing sequences of the signaling aptamers comprise or consist of the sequence 5′-GTCGTAAGCT-3′ (SEQ ID NO: 11) or 5′-AGCTTACGAC-3′ (SEQ ID NO: 12). In some embodiments, the first and second hybridizing sequences of the signaling aptamers comprise or consist of the sequences 5′-GTCGTAAGCT-3′ (SEQ ID NO: 11) and 5′-AGCTTACGAC-3′ (SEQ ID NO: 12).

In some embodiments, the hybridizing sequences of the signaling aptamers consist of the sequence 5′-GTCGTAAGT-3′ (SEQ ID NO: 9) or 5′-ACTTACGAC-3′ (SEQ ID NO: 10). In some embodiments, the TBA15 signaling aptamer contains a 3′ hybridizing sequence consisting of 5′-GTCGTAAGT-3′ (SEQ ID NO: 9). In some embodiments, the TBA15 signaling aptamer contains a 3′ hybridizing sequence consisting of 5′-ACTTACGAC-3′ (SEQ ID NO: 10). In some embodiments, the TBA29 signaling aptamer contains a 5′ hybridizing sequence consisting of 5′-GTCGTAAGT-3′ (SEQ ID NO: 9). In some embodiments, the TBA29 signaling aptamer contains a 5′ hybridizing sequence consisting of 5′-ACTTACGAC-3′ (SEQ ID NO: 10). In some embodiments, the TBA15 signaling aptamer contains a 3′ hybridizing sequence consisting of 5′-GTCGTAAGT-3′ (SEQ ID NO: 9) and the TBA29 signaling aptamer contains a 5′ hybridizing sequence consisting of 5′-ACTTACGAC-3′ (SEQ ID NO: 10). In some embodiments, the TBA15 signaling aptamer contains a 3′ hybridizing sequence consisting of 5′-ACTTACGAC-3′ (SEQ ID NO: 10) and the TBA29 signaling aptamer contains a 5′ hybridizing sequence consisting of 5′-GTCGTAAGT-3′ (SEQ ID NO: 9).

In some embodiments, a signaling aptamer contains a linker between the protein binding region (aptamer) and the hybridizing sequence. In some embodiments, the linker is a flexible linker. The linker can be a nucleobase linker or a non-nucleobase linker. A non-nucleobase linker can be, but is not limited to, a PEG or an aliphatic chain. In some embodiments, the linker is a PEG group. The PEG group can be (PEG)ₙ, wherein n is an integer from 1 to 20. In some embodiments, n is 1, 2, 3, 4, 5, 6, 7, 8, 9, 10, 11, 12, 13, 14, 15, 16, 17, 18, 19, or 20. In some embodiments, n is 6.

In some embodiments, a first signaling aptamer containing a first hybridizing sequence and a fluorescent label is paired with a second signaling aptamer containing a second hybridizing sequence complementary to the first hybridizing sequence and a quencher that reduces or alters fluorescence emitted by the fluorescent label.

In some embodiments, the kits further contain heparin. The heparin can be a naturally occurring heparin or a synthetic heparin. The heparin can be an unfractionated heparin or a low-molecular-weight heparin.

In some embodiments, the kits further contain heparin linked to a support. The heparin linked to a support can be, but is not limited to, a heparin bead. The heparin linked to the solid support can be used to purify any ATIII present in the sample from one or more other components in the sample.

In some embodiments, the kits contain suitable assay containers (e.g., sample tubes or wells) to determine ATIII in the presence and absence of the heparin. In some embodiments, the kits contain suitable assay containers to determine initial rate of signal production in the presence and absence of the heparin. Measuring the fluorescence signal in the presence and absence of heparin can be used to correct for proteins other than ATII in the sample that may disrupt the aptamer-thrombin complex. Proteins in the sample other than ATIII that disrupt the aptamer-thrombin complex could result in the assay overestimating the amount or concentration of ATIII in the sample. Because heparin selectively activates ATIII towards the aptamer complex, signal is higher at earlier times when ATIII is activated with heparin than when no heparin is present. By adding heparin to the aptamer assay and comparing the initial rate in the presence of heparin with the initial rate in the absence of heparin, any change in the initial rate of signal production comes from heparin-activated-ATIII. The change in initial rate between signal measured in the presence of heparin and the signal measured in the absence of heparin is correlated with ATIII concentration in the blood.

### D. Compositions

In some embodiments, thrombin-signaling aptamer complexes are described. Thrombin-signaling aptamer complexes comprise thrombin bound by the described first and second signaling aptamers. In some embodiments, the thrombin-signaling aptamer complexes are in a composition. The composition can contain one or more components that facilitate detection or measurement of fluorescence.

Thrombin can be prepared by a variety of methods known in the art, and the term “thrombin” is not intended to imply a particular method of production. The thrombin can be purified from a naturally occurring source, recombinant, synthetic, or synthesized from cells, such as bacteria, insect, yeast or mammalian cells, in culture. Thrombin can be purified from plasma. Both human and non-human thrombins can be used within the present invention. In some embodiments, the thrombin is human thrombin.

The thrombin-signaling aptamer complex can be provided in a solution or a lyophilized powder or cake. The thrombin-signaling aptamer complex can also be provided on a solid support, such as a test strip. In some embodiments, the signaling aptamers can be provided in a solution at a concentration of about 100 nM to about 400 nM. In some embodiments, the signaling aptamers can be provided in a solution at a concentration of about 100 nm, about 200 nM, about 300 nm, or about 400 nM. In some embodiments, the signaling aptamers can be provided in a solution at a concentration of about 200 nM. In some embodiments, the thrombin-signaling aptamer complex can be provided in a solution at a concentration of about 100 nM to about 400 nM. In some embodiments, the thrombin-signaling aptamer complex can be provided in a solution at a concentration of about 100 nm, about 200 nM, about 300 nm, or about 400 nM. In some embodiments, the thrombin-signaling aptamer complex can be provided in a solution at a concentration of about 200 nM.

In some embodiments, the molar ratio of the first signaling aptamer to the second signaling aptamer is about 1:1. In some embodiments, the molar ratio of first and second signaling aptamers to thrombin is about 1:1:2 to 2:2:1 (first signaling aptamer:second signaling aptamer:thrombin). In some embodiments, the molar ratio of first and second signaling aptamers to thrombin is about 1:1:1 (first signaling aptamer:second signaling aptamer:thrombin). In some embodiments, the molar ratio of TBA15 signaling aptamer to TBA29 signaling aptamer is about 1:1. In some embodiments, the molar ratio of the TBA15 signaling aptamer and TBA29 signaling aptamer to thrombin is about 1:1:2 to 2:2:1 (TBA15 signaling aptamer:TBA29 signaling aptamer:thrombin). In some embodiments, the molar ratio of TBA15 signaling aptamer and TBA29 signaling aptamer to thrombin is about 1:1:1 to 1:1:1.25 (TBA15 signaling aptamer:TBA29 signaling aptamer:thrombin). In some embodiments, the molar ratio of TBA15 signaling aptamer and TBA29 signaling aptamer to thrombin is about 1:1:1 (TBA15 signaling aptamer:TBA29 signaling aptamer:thrombin).

When a sample containing ATIII is added to a thrombin-signaling aptamer complex or a composition containing the thrombin-signaling aptamer complex, the ATIII binds to the thrombin, leading to dissociation of thrombin-signaling aptamer complex and an increase in fluorescence from the fluorescent label present on one of the signaling aptamers. The increase in fluorescence is proportional to the amount of ATIII in the sample. Dissociation of thrombin-signaling aptamer complex includes dissociation of the first signaling aptamer, the second signaling aptamer, or both signaling aptamers from the thrombin. In some embodiments, ATIII binding to thrombin dissociates the first signaling aptamer.

When a sample containing ATIII is added to a thrombin-signaling aptamer complex or a composition containing the thrombin-signaling aptamer complex in the presence of heparin, the heparin activates the ATIII to bind to the thrombin, leading to dissociation of thrombin-signaling aptamer complex and an increase in fluorescence from the fluorescent label present on one of the signaling aptamers. The initial rate of increase of fluorescence is proportional to the amount of ATIII in the sample. Dissociation of thrombin-signaling aptamer complex includes dissociation of the first signaling aptamer, the second signaling aptamer, or both signaling aptamers from the thrombin. In some embodiments, ATIII binding to thrombin dissociates the first signaling aptamer.

Heparin can be prepared by a variety of methods known in the art, and the term heparin is not intended to imply a particular method of production or a particular molecular weight of heparin. The heparin can be purified from a naturally occurring source, synthetic, or synthesized from cells, such as bacteria, insect, yeast or mammalian cells, in culture. In some embodiments, the heparin can be provided as a solution. In some embodiments, the heparin is provided coated to an assay container. The assay container can be a tube, vial, multi-well plate or other microfluidic device.

In some embodiments, the heparin is linked to a solid support. The solid support can be, but is not limited to beads, magnetically attractable particles, nitrocellulose, nylon, glass, polyacrylate, mixed polymers, polystyrene, silane polypropylene, slides, and multiwell plates. In some embodiments, the sample is purified to remove one or more components of the sample from ATIII in the sample prior to contacting the sample with the thrombin-signaling aptamer complex. Purifying the sample can comprise isolating ATIII bound to the heparin from the sample using the solid support or washing components of the sample not bound to the heparin from the solid support.

In some embodiments, the level of ATIII in the sample is determining by comparing the increase in fluorescence with a standard curve. The standard curve can be obtained by determining the level of increase in fluorescence for various known concentration of ATIII when measured under the same conditions.

In some embodiments, the level of ATIII in the sample is determining by comparing the initial rate of increase in fluorescence in the presence of heparin with the initial rate of increase in the absence of heparin and comparing with a standard curve. The standard curve can be obtained by determining the level of increase in fluorescence for various known concentration of ATIII when measured under the same conditions.

### E. Kits

Also provided are kits comprising one or more reagents utilized in performing a method disclosed herein or kits comprising one or more compounds or compositions disclosed herein. Such kits may be diagnostic in nature. The kits of example embodiments provide a point-of-care testing system that obtains a result relatively quickly for the level of ATIII in a patient.

Kits will generally comprise a described pair of signaling aptamers or thrombin-signaling aptamer complexes in a suitable container or receptacle. The kits may also contain additional reagents or devises useful in determining the level of ATIII in a sample. Such additional reagents or devices can be, but are not limited to, one or more of: buffer, ATIII, syringe, and hypodermic needle. The kits can be used to quantify the level of ATIII in one or more samples. The containers can be formed from a variety of materials including, but not limited to, glass and pharmaceutically compatible plastics.

In some embodiments, a kit comprises one or more containers comprising one or more of any of the described signaling aptamers or complexes. In some embodiments, a kit contains or more test strips comprising one or more of any of the described signaling aptamers or complexes. In some embodiments, a kit contains a unit dosage, meaning a predetermined amount of a composition comprising, for example, any of the described signaling aptamers or complexes suitable for determining the level of ATIII in a sample, optionally, with one or more additional reagents. In some embodiments, the kit contains one or more control samples.

In some embodiments, the kits can further contain heparin. In some embodiments the heparin is linked to a support. The heparin linked to a support can be, but is not limited to, a heparin bead. In some embodiments, the kits contain multi-well plates in which as least some of the wells contain heparin. In some embodiments, the kits are provided with multi-well plates in which some of the wells are coated with heparin, and some of the wells are not coated with heparin. In some embodiments, the kits contain heparin-coated and heparin uncoated well suitable for analyzing the sample in the presence and absence of heparin.

In some embodiments, the signaling aptamers, thrombin-signaling aptamer complexes, and/or heparin are provided in a liquid. The liquid can be sterile or not sterile. In some embodiments, the signaling aptamers, thrombin-signaling aptamer complexes, and/or heparin are provided in a lyophilized form that can be reconstituted upon addition of an appropriate solvent. The solvent used for reconstitution can be provided in a separate container.

In some embodiments, the signaling aptamers, thrombin-signaling aptamer complexes, and/or heparin are as a solution, lyophilized powder or cake, or coating in a sample vessel. The sample vessel can be a tube, microplate well or other vessel suitable for performing the assay.

In some embodiments, a kit comprises a label, marker, package insert, bar code and/or reader indicating that the kit contents may be used determine the level of ATII in a sample. In some embodiments, a kit can contain instructional material which describes use of the kit to measure the level of ATIII in a sample.

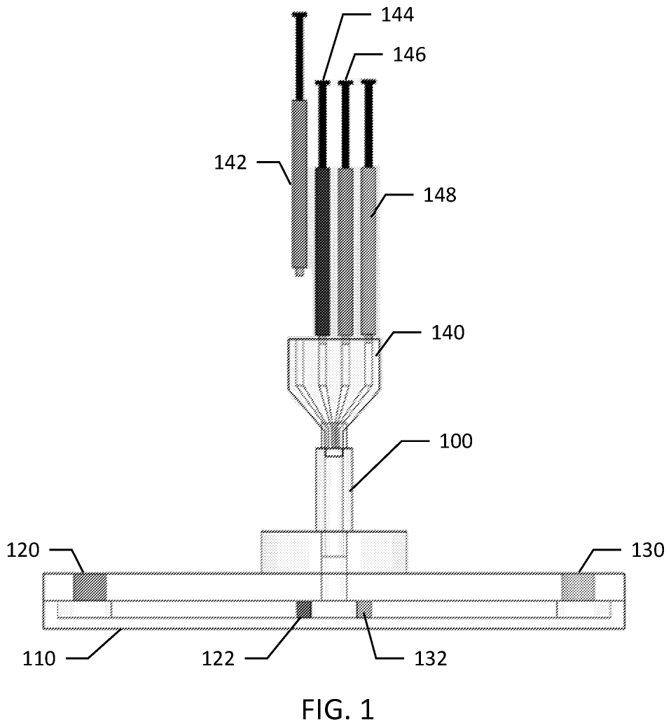

FIG. 1 illustrates an example system that may be part of a kit including a Heparin loaded bead assay. The system illustrated is an example embodiment which can be structurally changed while producing a similar result. As shown, the system includes a plurality of syringes configured to drive a fluid through the column 100 to the system substrate 110. The system substrate 110 includes a waste reservoir 120 and a fluorescence reservoir 130. A waste valve 122 and a fluorescence valve 132 control flow of fluid from the column 100 to the waste reservoir 120 and fluorescence reservoir 130, respectively.

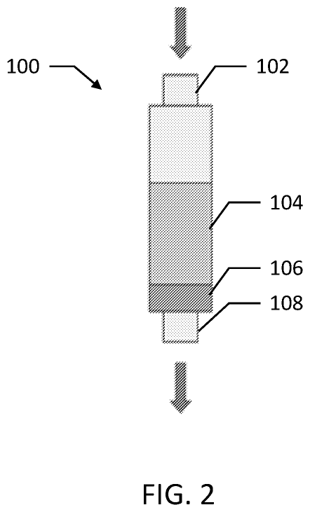

The column-based method of the illustrated embodiment includes syringes loaded into manifold 140. The syringes include a phosphate buffered saline (PBS) washing buffer syringe 142, a plasma syringe 144, a PBS syringe 146, and an aptamer assay syringe 148. The fluid from the syringes may be driven from the syringes into the manifold 140 and into the column 100 manually or with a mechanical device driving the respective plunger. A mechanical device driving fluid from the syringes may be beneficial for maintaining a consistent flow rate and pressure from the syringes through the column 100. The column 100, as further illustrated in FIG. 2, includes an inlet 102, a volume of compact sepharose beads 104 held within a chamber of the column, a membrane 106 to maintain the compact sepharose beads within the column, and an outlet 108. The liquid from the syringes is driven through the column to fulfill the washing, ATIII capture, washing, and assay step in that order.

The PBS washing buffer is first driven from syringe 142 through the inlet 102 of the cylinder 100, across the compact sepharose beads 104, and out the outlet 108 of the column. The membrane 106 retains the compact sepharose beads 104 within the column. The PBS washing buffer is received into the waste reservoir 120 responsive to waste valve 122 being in the open position and fluorescence valve 132 being in the closed position. The diluted plasma of syringe 144 is then driven through the column and across the sepharose beads and similarly is received into the waste reservoir 120. The PBS washing buffer of syringe 146 is then driven across the compact sepharose beads 106 and is received into waste reservoir 120. Once the second PBS washing buffer is driven through the column 100, the waste valve 122 is closed and the fluorescence valve 132 is opened. The aptamer assay syringe 148 is used to drive the incubated aptamer assay through the column 100 for fluorescence generation, and the fluid is received at the fluorescence reservoir 130 where the fluorescence is read to determine the ATIII level.

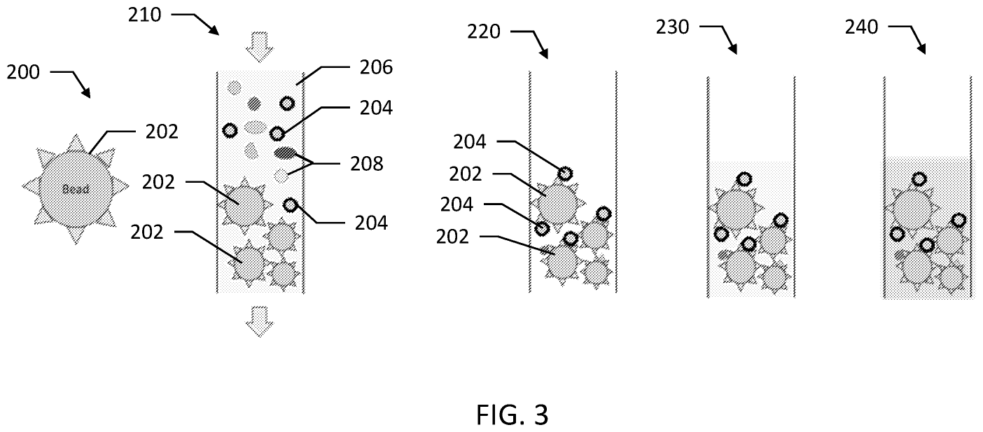

FIG. 3 illustrates the workflow of the heparin beads 202 in heparin bead-based detection of ATIII 204 in plasma 206. A heparin bead 202 is shown at 200 as a Heparin Sepharose bead. The heparin beads 202 are confined to the column (column 100 of FIG. 1) at 210, where the heparin beads 202 are washed with PBS washing buffer in the direction of the flow arrows. The washing and incubation of the heparin beads 202 with the diluted plasma 206 isolates the ATIII 204 and removes the interfering molecules 208 in the diluted plasma 206. After incubation with the plasma, ATIII 204 is captured on the surfaces of the heparin beads 202 via the interaction between ATIII and heparin as shown at 220. The heparin beads 202 are again washed by the PBS washing buffer at 230 to remove the off-target proteins followed by incubation with aptamer assay. ATIII captured on the beads will competitively trigger the release of aptamer-F into the solution, contributing to signal generation at 240. The determination of the fluorescence intensity reflects the ATIII concentration. The fluorescence intensity is read through the fluorescence well 130; however, all reactions occur in the column. Aptamer-F provides a high intensity of fluorescence to enable determination of the level of ATIII in the plasma.

Using the system described above with respect to FIGS. 1-3 provides a method of testing the level of ATIII in a patient. This process can be performed in a point-of-care setting with the results obtained relatively quickly as compared to traditional lab-based methods. The process developed by the Applicant is uniquely capable of identifying the ATIII levels in a patient quickly such that any ATIII deficiencies can be identified and addressed quickly. The system of FIGS. X-Z provides a unique process through which the ATIII can be measured through fluorescence using a specific sequence of compounds driven through the column holding the heparin beads as detailed above.

### F. Methods

Described are methods of using the described compounds and compositions to detect and/or quantify the amount of ATIII in a sample. In some embodiments, the methods comprise: forming a thrombin-signaling aptamer complex comprising thrombin, a first signaling aptamer, and a second signaling aptamer, contacting the thrombin-signaling aptamer complex with a sample containing or suspected of containing ATIII, and measuring fluorescence emitted by the fluorescent label. In some embodiments, the methods comprise contacting a thrombin-signaling aptamer complex with a sample containing or suspected of containing ATIII and detecting or measuring an increase in fluorescence signal emitted by the fluorescent label. In some embodiments, the increase in fluorescence signal is proportional to the level of ATIII in the sample.

Described are methods of using the described compounds and compositions to detect and/or quantify the amount of ATIII in a sample. In some embodiments, the methods comprise: forming a thrombin-signaling aptamer complex comprising thrombin, a first signaling aptamer, and a second signaling aptamer, contacting the thrombin-signaling aptamer complex with a sample containing or suspected of containing ATIII both in the presence and absence of heparin, and measuring fluorescence emitted by the fluorescent label in the presence and absence of heparin. In some embodiments, the methods comprise contacting a thrombin-signaling aptamer complex with a sample containing or suspected of containing ATIII in the presence and absence of heparin and detecting or measuring an increase in fluorescence signal emitted by the fluorescent label in the presence and absence of heparin. In some embodiments, the initial rate of increase in fluorescence is determined, The initial rate of increase in fluorescence in the presence or heparin relative to the initial rate of increase of fluorescence in the absence of heparin is proportional to the level of ATIII in the sample.

Described are methods of using the described compounds and compositions to detect and/or quantify the amount of ATIII in a sample. In some embodiments, the methods comprise: contacting a sample containing or suspected of containing ATIII with heparin linked to a solid support, purifying any ATIII in the sample form one or more other components of the sample using the heparin linked to the solid support to form a purified sample, forming a thrombin-signaling aptamer complex comprising thrombin, a first signaling aptamer, and a second signaling aptamer, contacting the thrombin-signaling aptamer complex with the purified sample, and measuring fluorescence emitted by the fluorescent label. In some embodiments, the methods comprise contacting a thrombin-signaling aptamer complex with a purified sample containing or suspected of containing ATIII and detecting or measuring an fluorescence signal emitted by the fluorescent label wherein the purified sample is formed by incubating the sample with heparin linked to a solid support wherein any ATIII in the sample is bound by the heparin.

The described methods for determining the level of ATIII in a sample are rapid, simple, and accurate. The methods can be performed prior to, concurrent with, and/or subsequent to a medical procedure. Medical procedures include, but are not limited to, cardiac surgery, vascular surgery, heart-lung machine bypass, cardiac catheterizations, dialysis, extracorporeal membrane oxygenation, filtration procedures, and radiology procedures.

In some embodiments, described compounds, compositions, and methods can be used to determine the level of ATIII in a subject within about 10%, within about 9%, within about 8%, within about 7%, within about 6%, within about 5%, within about 4%, within about 3%, within about 2%, or within about 1%. In some embodiments, described compounds, compositions, and methods can be used to determine the level of ATIII in a subject within about 5%. In some embodiments, the methods can be used to measure the level of ATIII in a sample in less than or equal to 30 min, less than or equal to 25 min, less than or equal to 20 min, less than or equal to 15 min, or less than or equal to 10 min.

In some embodiments, the described methods are used to determine if a subject is ATIII deficient. In some embodiments, the described methods are used to determine a dosage of heparin and/or exogenous ATIII to administer to a patient.

In some embodiments, the level of ATIII measure in sample is compared with a predetermined control or level. The predetermined control or level can be derived from a population of subjects known to respond to heparin and/or known to be deficient in ATIII. In some embodiments, a value less than the predetermined control indicates the subject is deficient in ATIII and would benefit from administration of exogenous ATIII. In some embodiments, if the level ATII in the sample is lower than a predetermined level, then exogenous ATIII is administered to the subject. In some embodiments, a value higher than the predetermined control indicates the subject is not deficient in ATIII and is likely to respond to heparin treatment. In some embodiments, if the level ATII in the sample is higher than a predetermined level, then heparin is administered to the subject.

Described are methods of treating a patient with an anticoagulant comprising: obtaining a serum, plasma, or blood sample from the patient; contacting the sample with a described thrombin-signaling aptamer complex; measuring an increase in fluorescence; and determining a level of ATIII in the sample based on the increase in fluorescence; and administering to the patient heparin and/or exogenous ATIII based on the determined level of ATIII in the sample.

### G. Listing of Embodiments

1. A kit for determining the level of anti-thrombin (ATIII) in a subject, comprising:
(a) a first signaling aptamer comprising a first thrombin-specific aptamer, a first hybridization sequence, and a fluorescent label;
(b) a second signaling aptamer comprising a second thrombin-specific aptamer, a second hybridization sequence, and a quencher; and
(c) heparin,
wherein the first and second hybridization sequences are complementary to each other, and wherein fluorescence of the fluorescent label is quenched when the first and second thrombin-specific signaling aptamers are bound to thrombin.

2. The kit of embodiment 1, wherein the first thrombin-specific aptamer comprises TBA15.

3. The kit of embodiment 1 or 2, wherein the second thrombin-specific aptamer comprises TBA29.

4. The kit of any one of embodiments 1-3, wherein the first and second hybridization sequences are 6-10 nucleobases in length.

5. The kit of embodiment 4, wherein the first and second hybridization sequences are 9 nucleobases in length.

6. The kit of any one of embodiments 1-5, wherein the first hybridization sequence comprises the sequence 5′-GTCGTA-3′ and the second hybridization sequence comprises 5′-TACGAC-3′ or the first hybridization sequence comprise the sequence 5′-TACGAC-3′ and the second hybridization sequence comprises 5′-GTCGTA-3′.

7. The kit of embodiment 6, wherein the first hybridization sequence comprises the sequence 5′-GTCGTAAGT-3′ and the second hybridization sequences comprises the sequence 5′-ACTTACGAC-3′ or the first hybridization sequence comprises the sequence 5′-ACTTACGAC-3′ and the second hybridization sequences comprises the sequence 5′-GTCGTAAGT-3′.

8. The kit of any one of embodiments 1-7, wherein the first and/or second signaling aptamers comprises a linker, wherein the linker connects the thrombin-specific aptamer to the hybridization sequence.

9. The kit of embodiment 8, wherein the linker comprises polyethylene glycol (PEG).

10. The kit of embodiment 9, wherein the PEG is PEG6.

11. The kit of any one of embodiments 1-10, wherein the fluorescent label comprises FITC, fluorescein, hexachlorofluorescein, rhodamine, Carboxy-X-Rhodamine, tetramethylrhodamine, IAEDANS, EDANS, coumarin, BODIPY FL, lucifer yellow, eosine, erythrosine, Texas Red, or cyanine.

12. The kit of any one of embodiments 1-11, wherein the quencher comprises a DABCYL, a BLACK HOLE QUENCHER, or a TAMRA compound.

13. The kit of any one of embodiments 1-12, further comprising thrombin.

14. The kit of embodiment 13, wherein the thrombin, first signaling aptamer, and second signaling aptamer form a complex.

15. A method of determining the level of ATIII in a sample from a subject, comprising:
(a) contacting a first composition with a first portion of a sample from the subject wherein the first composition comprises heparin; a thrombin; a first signaling aptamer comprising a first thrombin-specific aptamer, a first hybridization sequence, and a fluorescent label; and a second signaling aptamer comprising a second thrombin-specific aptamer, a second hybridization sequence, and a quencher, wherein the first and second hybridization sequences are complementary to each other and form a duplex, and wherein fluorescence of the fluorescent label is quenched; and
(b) contacting a second composition with a second portion of the sample wherein the second composition comprises a thrombin; a first signaling aptamer comprising a first thrombin-specific aptamer, a first hybridization sequence, and a fluorescent label; and a second signaling aptamer comprising a second thrombin-specific aptamer, a second hybridization sequence, and a quencher, wherein the first and second hybridization sequences are complementary to each other and form a duplex, and wherein fluorescence of the fluorescent label is quenched, and wherein the second composition does not contain heparin; and
(c) measuring an initial rate of increase in fluorescence of the first composition and an initial rate of increase in fluorescence of the second composition,
wherein the initial rate of increase of fluorescence of the first composition relative to the initial rate of increase of fluorescence of the second composition is proportional to the level of ATIII in the sample

16. The method of embodiment 17, wherein the sample comprises a serum sample, blood sample, or plasma sample.

17. A method of treating a patient with an anticoagulant comprising:
(a) determining the level of ATIII in the patient as in claim 15; and
(b) administering to the patient heparin and/or exogenous ATIII based on the level of ATIII in the sample determined in step (a).

18. A kit or embodiment 1, wherein the kit further comprises a microfluidic device, wherein at least one of the wells of the microfluidic device is coated with the heparin and at least one of the wells of the microfluidic device is not coated with the heparin.

19. A method of determining the level of ATIII in a subject, comprising:
(a) contacting a sample from the subject with heparin linked to a solid support,
wherein any ATIII in the sample binds to the heparin;
(b) purifying the sample to remove one or more components of the sample that do not bind to the heparin to form a purified sample;
(c) contacting the purified sample with a thrombin-signaling aptamer complex; and
(d) measuring fluorescence wherein the level of fluorescence is proportional to the level of ATIII in the sample.

### EXAMPLES

### Example 1. Exemplary Signaling Aptamers

TBA15-F6
(SEQ ID NO: 13)
(5′-GGTTGGTGTGGTTGG-(PEG)₆-GTCGTA-FITC-3′)
TBA29-D6
(SEQ ID NO: 14)
(5′-DABCYL-TACGAC-(PEG)₆-AGTCCGTGGTAGGGCAGGTTGGGGT
GACT-3′),
TBA15-F7
(SEQ ID NO: 15)
(5′-GGTTGGTGTGGTTGG-(PEG)₆-GTCGTAT-FITC-3′)
TBA29-D7
(SEQ ID NO: 16)
(5′-Dabcyl-ATACGAC-(PEG)₆-AGTCCGTGGTAGGGCAGGTTGGGG
TGACT-3′)
TBA15-F8
(SEQ ID NO: 17)
(5′-GGTTGGTGTGGTTGG-(PEG)₆-GTCGTAGT-FITC-3′)
TBA29-D8
(SEQ ID NO: 18)
(5′-Dabcy1-ACTACGAC-(PEG)₆-AGTCCGTGGTAGGGCAGGTTGGG
GTGACT-3′)
TBA15-F9
(SEQ ID NO: 19)
(5′-GGTTGGTGTGGTTGG-(PEG)₆-GTCGTAAGT-FITC-3′)
TBA29-D9
(SEQ ID NO: 20)
(5′-Dabcyl-ACTTACGAC-(PEG)₆-AGTCCGTGGTAGGGCAGGTTGG
GGTGACT-3′)
TBA15-F10
(SEQ ID NO: 21)
(5′-GGTTGGTGTGGTTGG-(PEG)₆-GTCGTAAGCT-FITC-3′)
TBA29-D10
(SEQ ID NO: 22)
(5′-Dabcyl-AGCTTACGAC-(PEG)₆-AGTCCGTGGTAGGGCAGGTTG
GGGTGACT-3′)

### Example 2. Reagents

All the DNAs (Integrated DNA technologies) were stored at −20° C. with Tris-HCl buffer (Tris-acetate 20 mM, KCl 5 mM, NaCl 140 mM, MgCl₂ 1 mM, CaCl₂ 1 mM, pH=7.5).

Human α-thrombin (Haematologic Technologies, Inc.) was dissolved in the 50% glycerol/water (v/v) and stored at −20° C.

Antithrombin (Grifols Therapeutics Inc.) was purchased as a powder and mixed with 10 mL deionized water, and divided into 10 vials, which were all stored at 4° C. The concentration was >50 times normal human antithrombin concentration. The normal concentration of Antithrombin III in plasma is 2.3 μM, with 3-day half-life.

Heparin sodium salt (Santa Cruz biotechnology) was stored at 4° C.

Albumin from human serum (Sigma), was stored at 4° C.

### Example 3. Formation of Thrombin-Signaling Aptamer Complex

Thrombin was prepared at a concentration of 200 nM. The ratio between the TBA15 signaling aptamer and the TBA29 signaling aptamer was 1:1. The signaling aptamers were prepared at concentrations of 100 nM, 200 nM, 300 nM and 400 nM. Thrombin-signaling aptamer complexes were formed by combining the thrombin with different concentrations of signaling aptamers. For each, the thrombin and signaling aptamer solutions were incubated together in the Tris-HCl buffer (Tris-acetate 20 mM, KCl 5 mM, NaCl 140 mM, MgCl₂ 1 mM, CaCl₂ 1 mM, pH 7.5) with a total volume of 100 μL at room temperature for 30 min. For controls, TBA15 signaling aptamer alone (control 1) or TBA15 signaling aptamer+TBA29 signaling aptamer (control 2) were used. Fluorescence spectroscopy was used for detection.

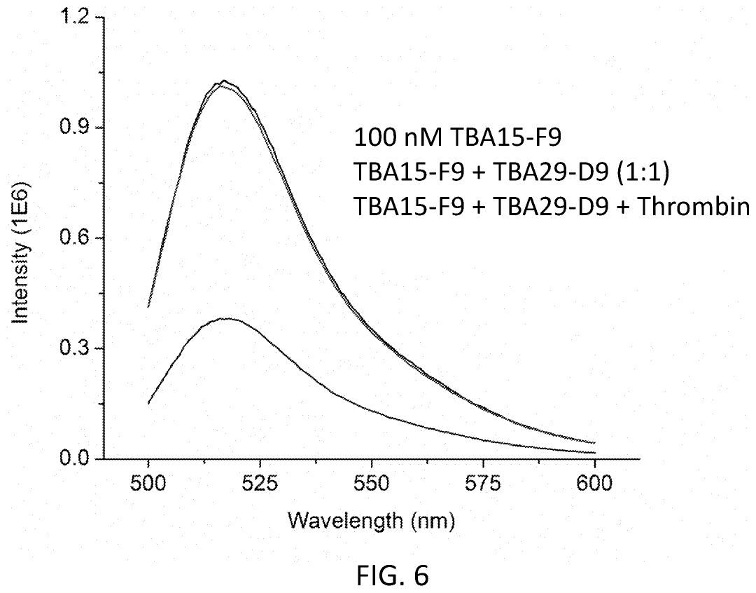

As shown in FIG. 6, at 100 nM concentration, peak fluorescence intensity was 3.80×10⁵ for the thrombin-signaling aptamer complex and set as set as I_{FDTHR}. Peak fluorescence intensity for TBA15 signaling aptamer alone was 1.029×10⁶ and set as I₀. Peak fluorescence intensity for the TBA15-F9+TAB29-D9 signaling aptamers was 1.011×10⁶ and set as I_{FD}. The fluorescence intensity of the TBA15 signaling aptamer alone was normalized as 100%. I_{FD}/I₀ was 98.25%. The relative fluorescence intensity of the thrombin-signaling aptamer complex (I_{FDTHR}/I₀) was 36.92%.

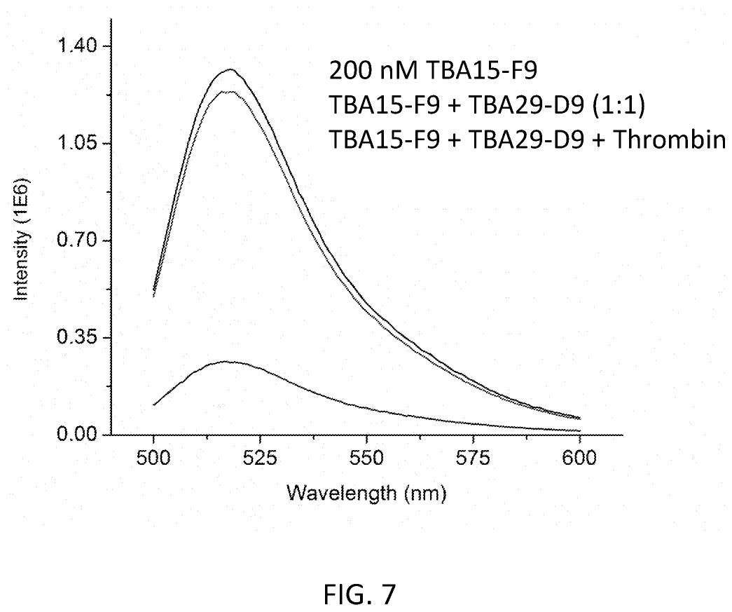

As shown in FIG. 7, at 200 nM concentration, peak fluorescence intensity for TBA15-F9 alone was 1.31×10⁶. Peak fluorescence intensity for TBA15-F9+TBA29-D9 was 1.23H10⁶ was 2.66×10⁵. Fluorescence intensity for TBA15-F9 alone was normalized as 100% and set as I₀. Fluorescence intensity for TBA15-F9+TBA29-D9 was set as I_{FD}. Then the I_{FD}/I₀ was 93.89%. The relative fluorescence intensity of the thrombin-signaling aptamer complex (I_{FDTHR}/I₀) was 20.23%.

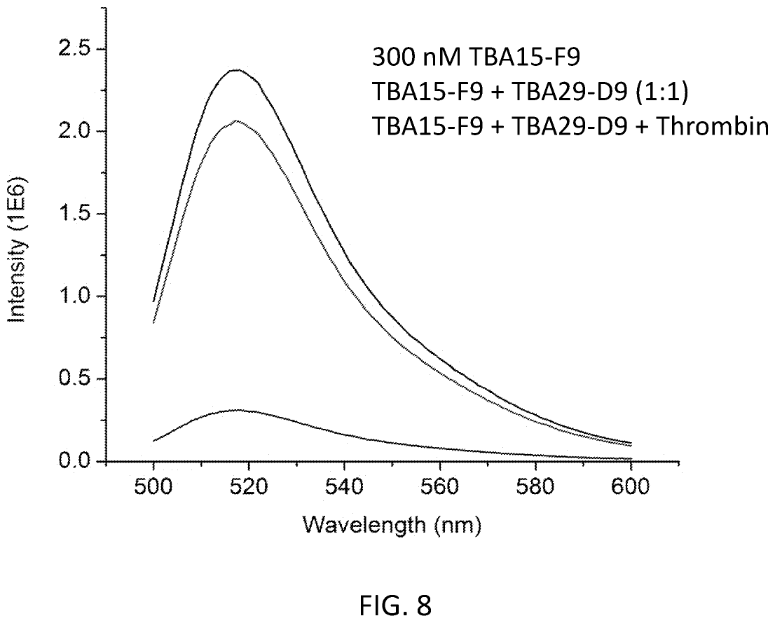

As shown in FIG. 8, at 300 nM concentration, peak fluorescence intensity for TBA15-F9 alone was 2.36×10⁶. Peak fluorescence intensity for TBA15-F9+TBA29-D9 was 2.06×10⁶. Peak fluorescence intensity for thrombin-signaling aptamer complex was 3.11×10⁵. Fluorescence intensity of TBA15-F9 alone was normalized as 100% and set as I₀. Fluorescence intensity for TBA15-F9+TBA29-D9 was set as I_{FD}. Relative fluorescence intensity for TBA15-F9+TBA29-D (I_{FD}/I₀) was 87.29%. The relative fluorescence intensity of the thrombin-signaling aptamer complex (I_{FDTHR}/I₀) was 13.17%.

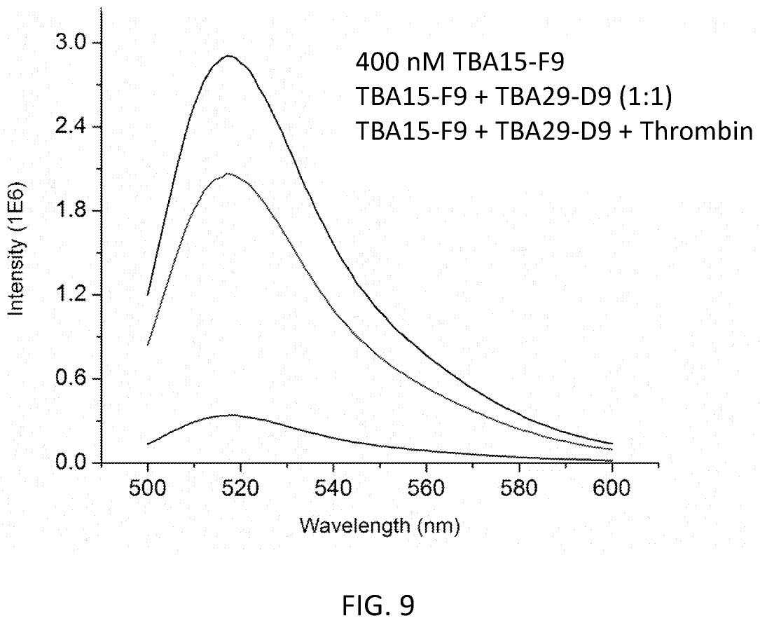

As shown in FIG. 9, at 400 nM concentration, peak fluorescence intensity for TBA15-F9 alone was 2.90×10⁶. Peak fluorescence intensity for TBA15-F9+TBA29-D9 was 2.06×10⁶. Peak fluorescence intensity for thrombin-signaling aptamer complex was 3.36×10⁵. Fluorescence intensity of TBA15-F9 alone was normalized as 100% and set as I₀. Fluorescence intensity for TBA15-F9+TBA29-D9 was set as I_{FD}. Relative fluorescence intensity for TBA15-F9+TBA29-D (I_{FD}/I₀) was 71.03%. The relative fluorescence intensity of the thrombin-signaling aptamer complex (I_{FDTHR}/I₀) was 11.58%.

Based on these experiments, a 1:1:1 ratio of thrombin to TBA15 signaling aptamer to TBA29 signaling aptamer was selected for additional experimentation (FIGS. 6-9).

### Example 4. Hybridization Sequences

Five hybridization sequences (with complementary hybridization sequences) were tested for use with the signaling aptamers. The hybridization sequences were those as described for TBA15-F6+TBA29-D6, TBA15-F7+TBA29-D7, TBA15-F8+TBA29-D8, TBA15-F9+TBA29-D9, and TBA15-F10+TBA29-D10. Controls were as described above.

The TBA15 signaling aptamers and the TBA29 signaling aptamers were prepared at concentration of 200 nM and combined at a ratio of 1:1. Thrombin-signaling aptamer complexes were formed by combining the signaling aptamers with varying concentrations of thrombin. The ratio of aptamers to thrombin was (1:1:0.1 to 1:1:1.5). For each, the thrombin and signaling aptamer solutions were incubated together in the Tris-HCl buffer with a total volume of 100 μL at room temperature for 30 min. For controls, TBA15 signaling aptamer alone or TBA15 signaling aptamer+TBA29 signaling aptamer were used. Fluorescence spectroscopy was used for detection.

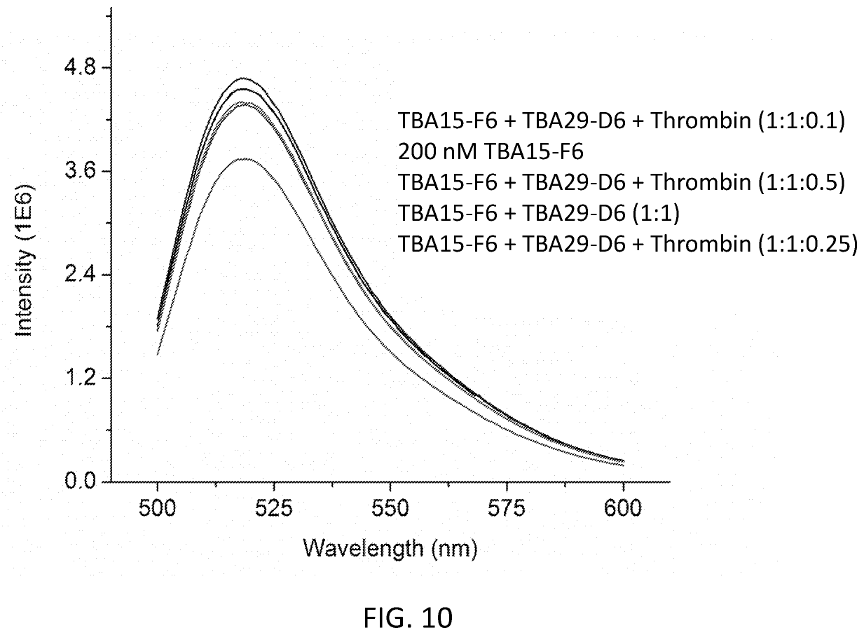

For aptamers TBA15-F6 and TBA29-D6, no specific trend was observed for the different concentrations of thrombin (FIG. 10). No specific trend was observed for the different concentrations of thrombin. The data suggest the two aptamers didn't bind to the thrombin and/or the hybridization sequences of the two signaling aptamers failed to form a duplex.

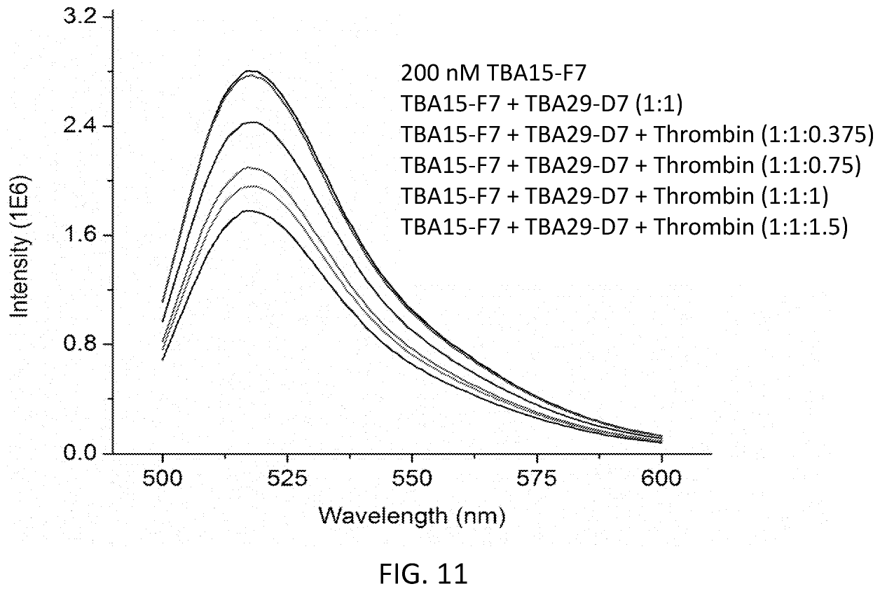

For aptamers TBA15-F7 and TBA29-D7, fluorescence intensity decreased and thrombin concentration increased (FIG. 11). Peak fluorescence intensity for TBA15-D7 alone was 2.806×10⁶, normalized at 100%, and set as I₇₀. Peak fluorescence intensity for TBA15-F7+TBA29-D7 (1:1) was 2.768×10⁶, set as I_{FD7}. Peak fluorescence intensity of TBA15-F7+TBA29-D7+thrombin (1:1:1) was 1.95×10⁶, set as I_{FDTHR7}. I_{FD7}/I₇₀ was =98.64%. I_{FDTHR7}/I₇₀=69.49%. The data indicate the two signaling aptamers bound to the thrombin and the hybridization sequences formed a duplex. The fluorescence of FITC was quenched by the dabcyl. For TBA15-F7+TBA29-D7 in the solution, fluorescence did not appear to be quenched, indicating binding to thrombin was necessary for duplex formation by the hybridization sequences and quenching of fluorescence.

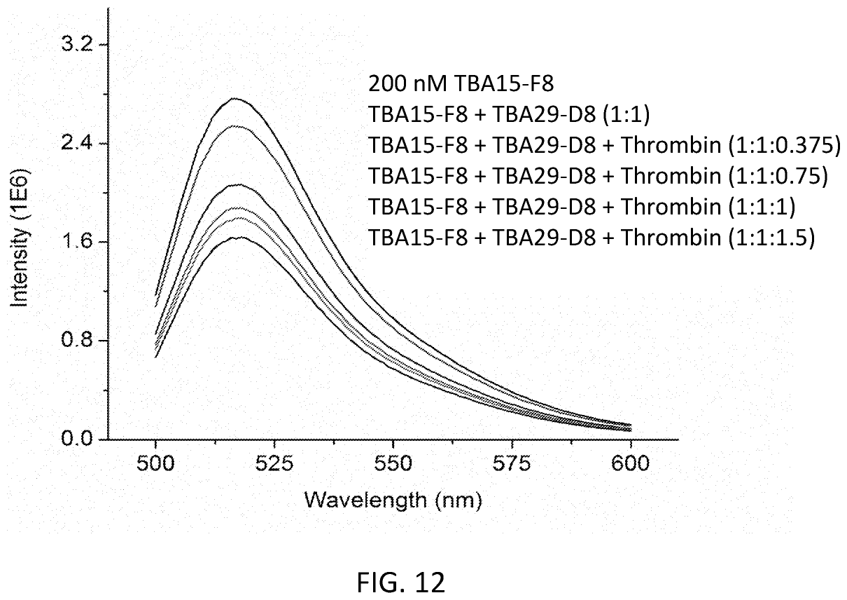

For aptamers TBA15-F8 and TBA29-D8, fluorescence intensity decreased and thrombin concentration increased (FIG. 12). Peak fluorescence intensity for TBA15-D8 alone was 2.76×10⁶, normalized at 100%, and set as I₈₀. Peak fluorescence intensity for TBA15-F8+TBA29-D8 (1:1) was 2.54×10⁶, set as I_{FD8}. Peak fluorescence intensity of TBA15-F8+TBA29-D8+thrombin (1:1:1) was 1.79×10⁶, set as I_{FDTHR8}. I_{FD8}/I₈₀ was =92.03%. I_{FDTHR8}/I₈₀=64.86%. The data indicate the two signaling aptamers bound to the thrombin and the hybridization sequences formed a duplex. The fluorescence of FITC was quenched by the dabcyl. Compared to the TBA15-F6 and TBA15-F7, improved quenching was observed with TBA15-F8 and TBA29-F8 in the presence of thrombin. A slight decrease in I_{FD}8/I₈₀ indicates TBA15-F8 and TBA29-F8 can interact, though weakly, in the absence of thrombin.

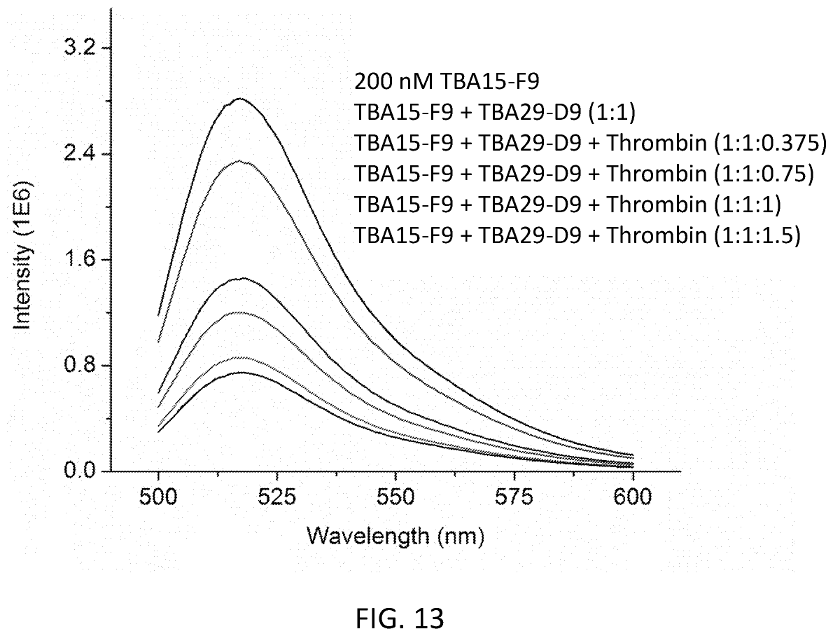

For aptamers TBA15-F9 and TBA29-D9, fluorescence intensity decreased and thrombin concentration increased (FIG. 13). Peak fluorescence intensity for TBA15-D9 alone was 2.81×10⁶, normalized at 100%, and set as I₉₀. Peak fluorescence intensity for TBA15-F9+TBA29-D9 (1:1) was 2.35×10⁶, set as I_{FD9}. Peak fluorescence intensity of TBA15-F9+TBA29-D9+thrombin (1:1:1) was 0.85×10⁶, set as I_{FDTHR9}. I_{FD9}/I₉₀ was =83.63%. I_{FDTHR9}/I₉₀=30.25%. The data indicate the two signaling aptamers bound to the thrombin and the hybridization sequences formed a duplex. The fluorescence of FITC was quenched by the dabcyl. TBA15-F9 and TBA29-F9 exhibited improved quenching compared to signaling aptamers having 8 base hybridization sequences. As with TBA15-F8 and TBA29-F8, TBA15-F9 and TBA29-F9 showed some quenching in the absence of thrombin.

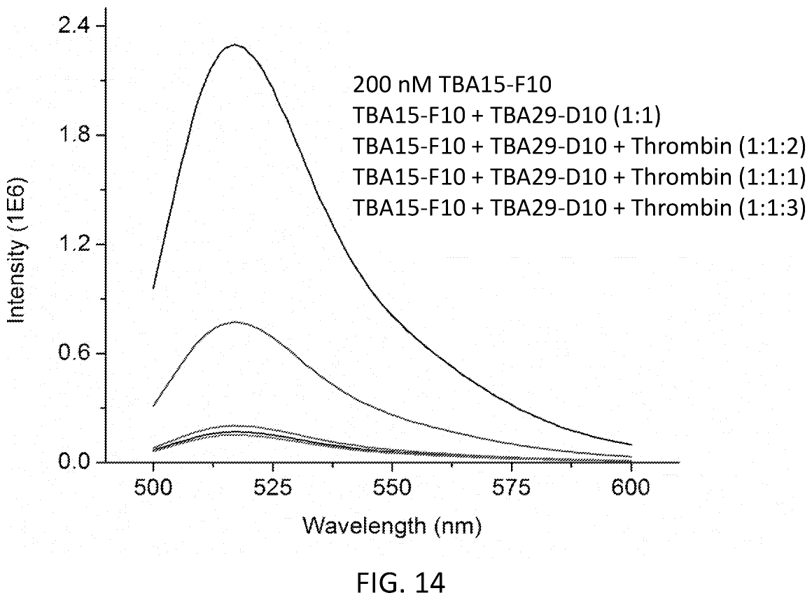

For aptamers TBA15-F10 and TBA29-D10, fluorescence intensity decreased and thrombin concentration increased (FIG. 14). Peak fluorescence intensity for TBA15-D10 alone was 2.30×10⁶, normalized at 100%, and set as I₁₀₀. Peak fluorescence intensity for TBA15-F10+TBA29-D10 (1:1) was 0.775×10⁶, set as I_{FD10}. Peak fluorescence intensity of TBA15-F10+TBA29-D10+thrombin (1:1:1) was 0.16×10⁶, set as I_{FDTHR10}. I_{FD10}/I₁₀₀ was =33.69%. I_{FDTHR10}/I₁₀₀=6.95%. The data indicate the two signaling aptamers bound to the thrombin and the hybridization sequences formed a duplex. The fluorescence of FITC was quenched by the dabcyl. TBA15-F10 and TBA29-F10 exhibited greater quenching compared to signaling aptamers having 9 base hybridization sequences. TBA15-F10 and TBA29-F10 also exhibited significant quenching in the absence of thrombin.

Based on the results, TBA15-F9+TBA29-D9 was used for additional experimentation.

### Example 5. Heparin Selectivity

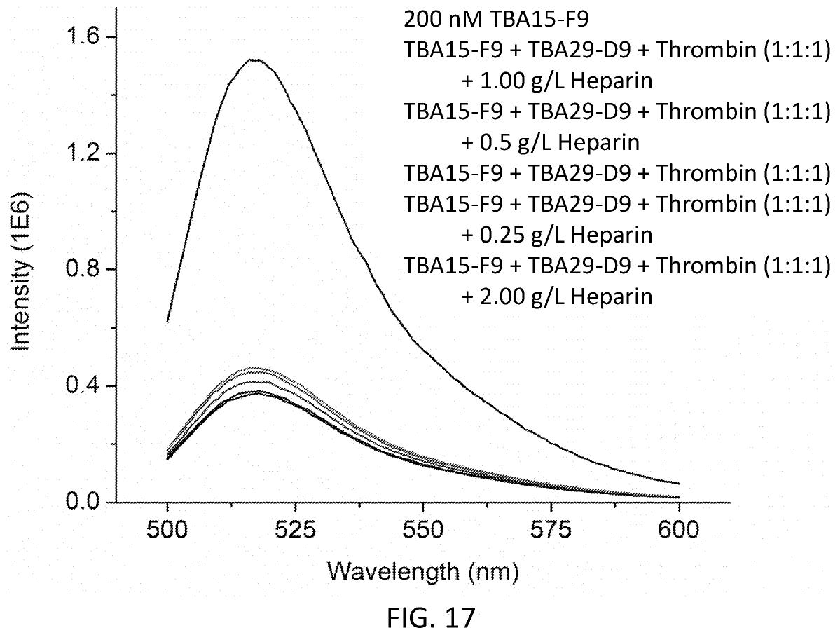

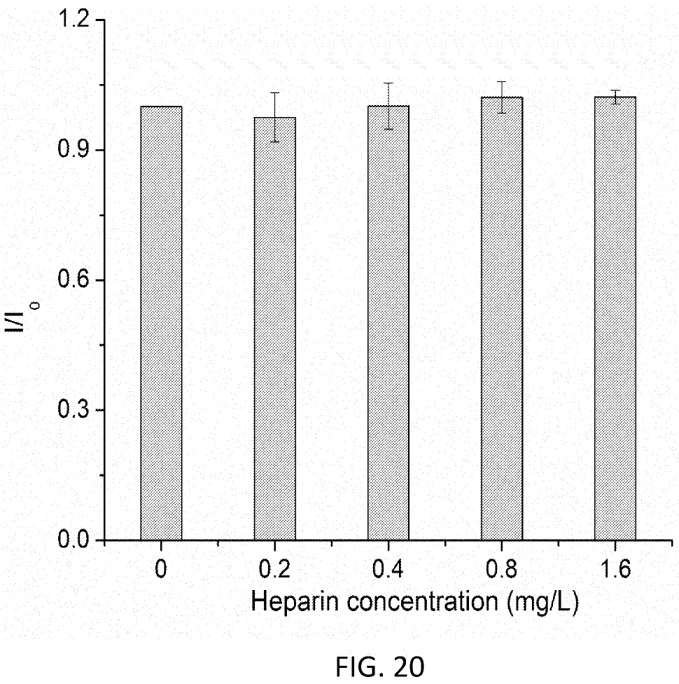

The ability of the thrombin-signaling aptamer complexes to accurately detect ATIII in the presence of heparin and human serum protein was tested. 200 nM TBA15-F9 only or 200 nM TBA15-F9+TBA29-D9+Thrombin (1:1:1) were used as controls. After complex formation, increasing concentrations of heparin (0.25 g/L to 2.00 g/L, FIG. 17; or 0.0 g/L to 1.6 g/L, FIG. 20) were added and incubated for 30 min at room temperature. The normal concentration of heparin in human plasma is 1.5-3.0 g/L. 1940 nM ATIII was then added and fluorescence spectroscopy was used to for detection. Fluorescence intensity with 1940 nM ATIII alone was normalized to 100%. Fluorescence intensity was not affected by the presence of heparin (FIGS. 17 and 20). The data indicate the thrombin-signaling aptamer complex does not react with heparin, and that the presence of heparin did not adversely affect ATIII detection.

### Example 6 Heparin Binding Antigen (HBA) Selectivity

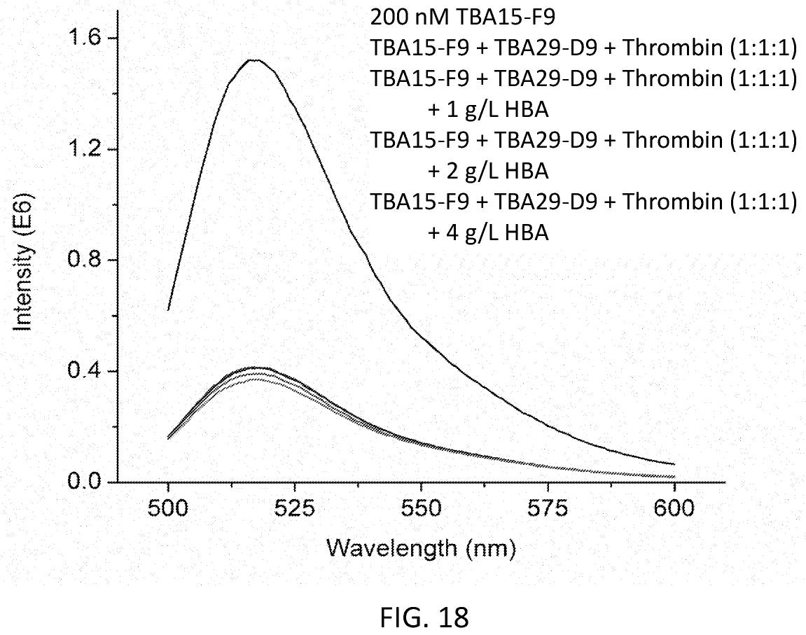

Similarly to heparin, the ATIII assay was also analyzed for HBA sensitivity. HBA is a protein created by certain bacteria that can interfere with heparin activity and heparin assays. 200 nM TBA15-F9, TBA29-D9 and thrombin (1:1:1) were incubated at room temperature for 30 min to form the thrombin-signaling aptamer complex. Various concentrations of HBA (1 g/L to 4 g/L) were then added to the complexes and incubated for 30 min at room temperature. ATIII was then added to the reactions and detected by fluorescence spectroscopy. Fluorescence intensity was not affected by the presence of heparin biding antigen, indicating the thrombin-signaling aptamer complex does not react with heparin and the presence of HBA does not adversely affect ATIII detection (FIG. 18).

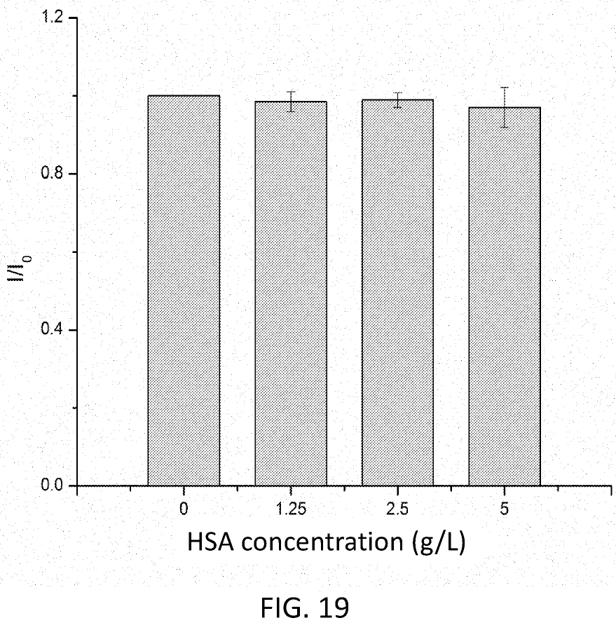

Similarly, the ATIII assay was also analyzed for human serum albumin sensitivity. Albumin is the most ubiquitous protein circulating in plasma and affects surface activity of the glycocalyx on endothelial cells. Albumin interacts with glucose-aminoglycans, specifically heparin. Sensitivity to HSA was tested in the same manner as sensitivity to HBA. After complex formation, 1940 nM ATIII and increasing concentrations of HSA were added. Fluorescence intensity with 1940 nM ATIII alone was normalized to 100%. HSA presence did not affect fluorescence intensity, indicating HBA does not adversely affect the reaction and the presence of HSA does not adversely affect ATIII detection (FIG. 19).

### Example 7. Antithrombin Response Curve

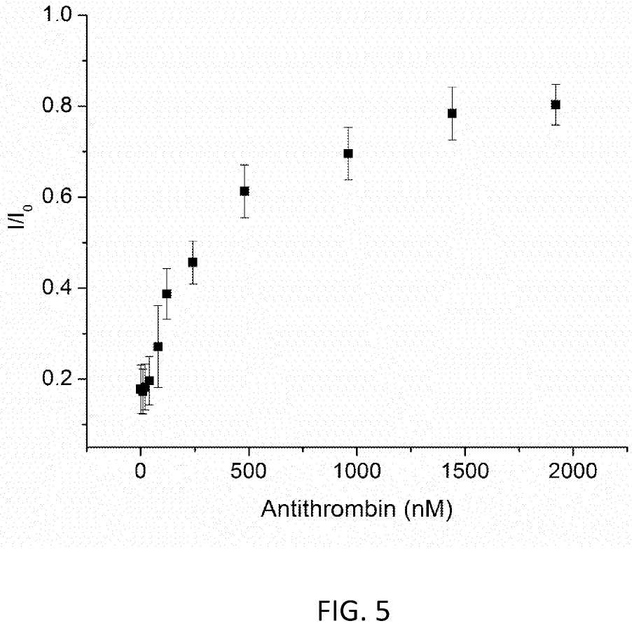

Varying concentration of ATIII were used to examine the utility of the thrombin-signaling aptamer complexes in quantifying ATIII. Increasing concentrations of ATIII were added to thrombin-signaling aptamer complex and fluorescence detected by fluorescence spectroscopy. There are 3 replicates for each sample (FIG. 5).

The antithrombin was titrated into the TBA15-F9-TBA29-D9-Thrombin complex. The complex concentration was 200 nM. The ratio of TBA15-F9:TBA29-D9:Thrombin was 1:1:1. The antithrombin concentration increased from 10 nM to 1960 nM. Fluorescence intensity of the TBA15-F9 alone control was normalized as 100% set as I₀. I/I₀ set in FIG. 20 y-axis means the intensity of each sample compared to the intensity of FITC in the solution only. Each concentration was repeated for 3 times. The data show the signaling aptamer complexes can be used to quantify ATIII in a sample.

### Example 8. Combination of Antithrombin (ATIII) and Heparin

The ability of heparin to promote the reaction between the antithrombin and heparin was analyzed. 200 nM TBA15-F9, TBA29-D9 and thrombin (1:1:1) were incubated for 30 min at room temperature to form thrombin-signaling aptamer complexes. Increasing concentrations of antithrombin were added to the complexes and incubated at room temperature for 30 min and fluorescence detected by fluorescence spectroscopy. There were 3 replicates for each sample.

### Example 9. Dynamic Study

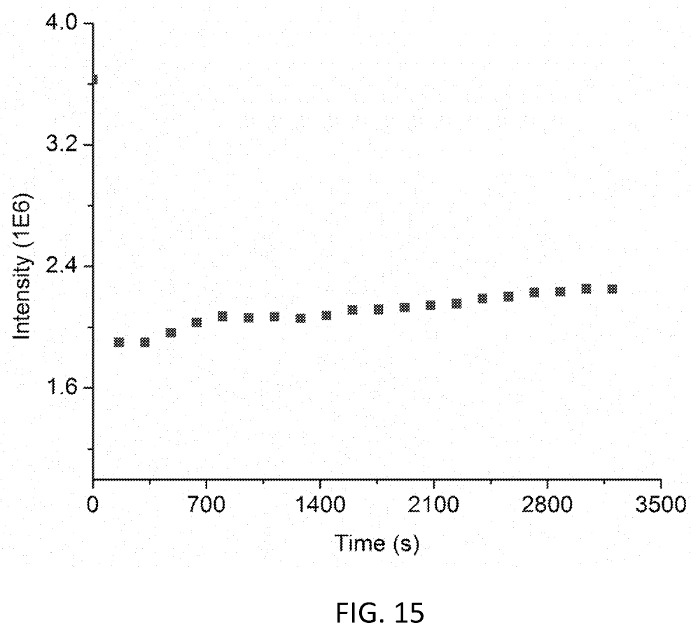

A. TBA15-F9+TBA29-D9+Thrombin dynamics. The 10 μL of 2 μM TBA15-F9 and 10 μL of 2 μM, thrombin were prepared and combined in 78 μL Tris-HCl. 10 μL of 2 μM TBA29-D9 was added to the solution while monitoring fluorescence. The reaction was complete in about 160 seconds (FIG. 15).

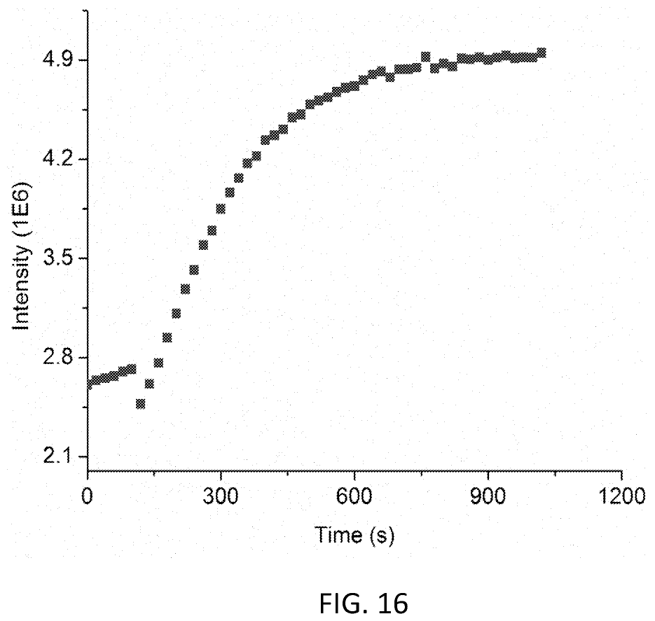

B. Complex+antithrombin dynamics. The 12 μL of 2 μM TBA15-F9, 12 μL of 2 μM TBA29-D9 and 12 μL of 2 μM thrombin (1:1:1 ratio) were combined in 76 μL Tris-HCl for 30 min at room temperature. 8 μL of 28.47 μM (1940 nM) ATIII was then titrated into the solution while monitoring fluorescence. The reaction was complete in about 600 seconds (FIG. 16).

### Example 10. Kinetic Response of the Aptamer Assay in the Presence of Heparin

Antithrombin III was prepared at a concentration of 500 nM from 100 μM stock.

Heparin, at a concentration of 1920 μM was used to from various dilutions as indicated below:
5-fold dilution=384 μM heparin: 20 μL 1920 μM heparin+80 μL of water
10-fold dilution=192 μM heparin: 50 μL 384 μM heparin+50 μL of water.
20-fold dilution=96 μM heparin: 50 μL 192 μM heparin+50 μL of water.
50-fold dilution=38.4 μM heparin: 40 μL 96 μM heparin+60 μL of water.
100-fold dilution=19.2 μM heparin: 50 μL 38.4 μM heparin+50 μL of water.
1000-fold dilution=1.92 μM heparin: 10 μL 19.2 μM heparin+90 μL of water.
2000-fold dilution=0.96 μM heparin: 50 μL 1.92 μM heparin+50 μL of water.
4000-fold dilution=0.48 μM heparin: 50 μL 0.96 μM heparin+50 μL of water.

### Aptamer Solution:

| Component | Single reaction | Stock solution |
|---|---|---|
| Fluorophore labeled Aptamer (F) | 10 μL | 240 μL |
| Quencher conjugated Aptamer (Q) | 10 μL | 240 μL |
| (alpha) thrombin | 0.065 μL | 1.56 μL |
| 10 × PBS | 5 μL | 120 μL |
| Water | 15 μL | 360 μL |

### Procedure:

1. 5 μL (500 nM) ATIII was mixed with 5 μL water in the first and second reaction wells and served as the (no heparin) control.
2. In separate wells, 5 μL (500 nM) ATIII was mixed with 5 μL of each of 384 μM heparin, 192 μM heparin, 96 μM heparin, 38.4 μM heparin, 19.2 μM heparin, 1.92 μM heparin, 0.96 μM heparin, and 0.48 μM heparin.
3. 40 μL Aptamer solution was then added to each reaction well.
4. Fluorescence was measures over time as described above.

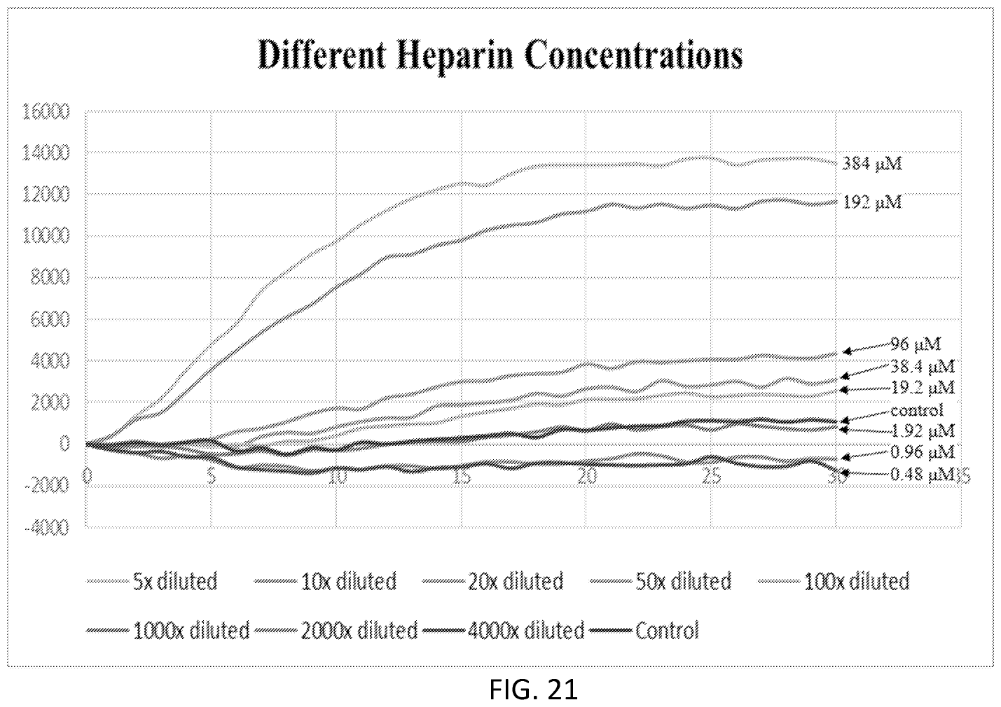

As shown in FIG. 21, increasing concentration of heparin in the assay more strongly activated AT III and produced faster kinetics of reaction when the aptamer solution was added. The control (no heparin) sample exhibited faster kinetics than the samples made using 1.92 μM, 0.96 μM, and 0.45 μM heparin. In a duplicate experiment, the control sample exhibited faster kinetics than the sample made using 96 μM heparin solution, indicating that heparin did not activate ATIII fast enough for it to react with the aptamer solution.

### Example 11. Effect of Heparin in Different ATIII Concentrations

Heparin was prepared at a concentration of 192 μM.

ATIII, at a concentration of 100 μM, was used to from various dilutions as indicated below:
20 μM: 200 μL 100 μM ATIII+800 μL water (final assay concentration=2 μM ATIII)
10 μM: 100 μL 100 μM ATIII+900 μL water (final assay concentration=1 μM ATIII)
5 μM: 500 μL 10 μM ATIII+500 μL water (final assay concentration=500 nM ATIII)
2 μM: 400 μL 5 μM ATIII+600 μL water (final assay concentration=200 nM ATIII)
1 μM: 200 μL 5 μM ATIII+800 μL water (final assay concentration=100 nM ATIII)

### Aptamer Solution:

| component | single reaction | stock solution |
|---|---|---|
| Fluorophore labeled Aptamer (F) | 10 μL | 260 μL |
| Quencher conjugated Aptamer (Q) | 10 μL | 260 μL |
| (alpha) thrombin | 0.065 μL | 1.696 μL |
| 10 × PBS | 5 μL | 130 μL |
| Water | 15 μL | 390 μL |

### Procedure:

1. 5 μL (192 μM) heparin was mixed with 10 μL of water in the first and second reaction well and served as the (no ATIII) control.
2. In separate wells, 5 μL (192 μM) heparin was mixed with 5 μL of each of 20 μM, 10 μM, 5 μM, 2 μM, and 1 μM ATIII.
   In separate wells, 5 μL water was mixed with 5 μL of each of 20 μM, 10 μM, 5 μM, 2 μM, and 1 μM ATIII.
3. 40 μL Aptamer solution was then added to each reaction well.
4. Fluorescence was measures over time as described above.

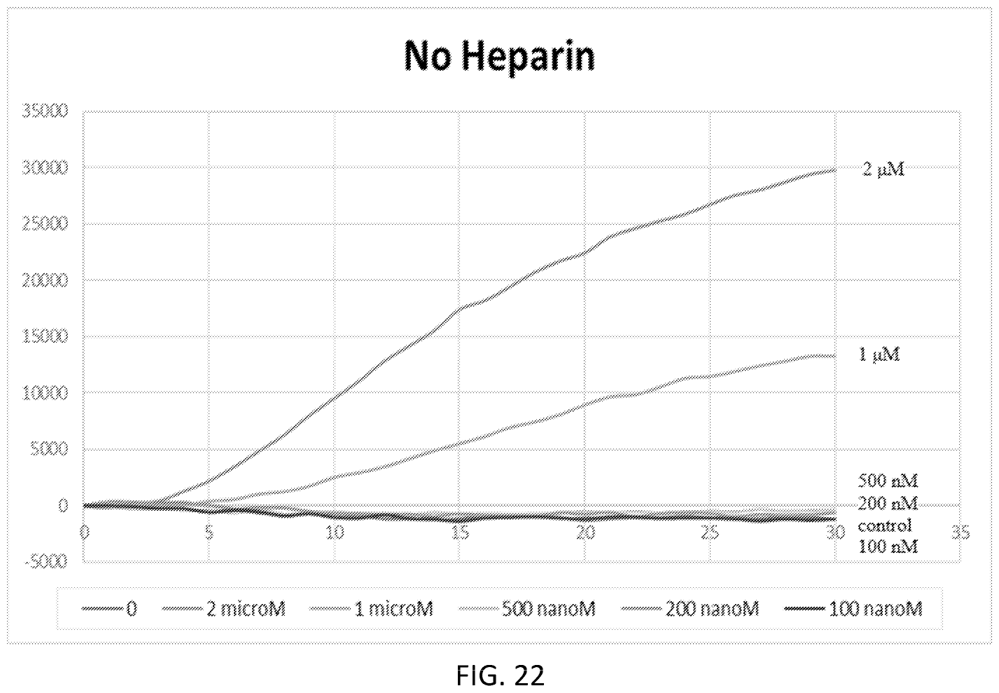

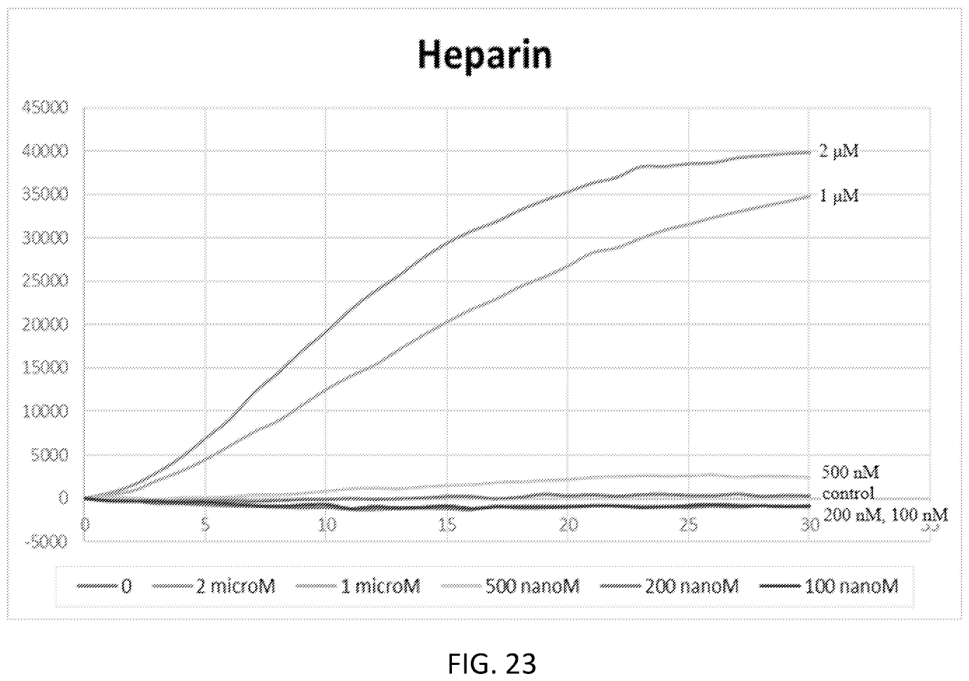

The data was normalized and graphed as shown in FIGS. 22 and 23. Fluorescence signal was higher in the presence of heparin (compare FIG. 22 (no heparin) with FIG. 23 (with heparin), confirming heparin activation of ATIII. The limit of detection of ATIII in the absence of heparin was 1 μM. The limit of detection of ATIII in the presence of heparin was 500 nM (0.5 μM).

### Example 12. Heparin-Containing ATII Assay Device

A. Microplates are prepared with various sample wells coated with or without heparin. Optionally, positive control wells are coated with or without heparin and with or without various predetermined amounts of ATIII. Optionally, positive control samples containing various predetermined amounts of ATIII are be provided. Samples from one or more subjects are added to the wells the quantity of ATIII in the samples is determined as described. The observed difference in initial rate of signal output between the wells correlates with ATIII concentration.

B. The approach described above be incorporated into various other kits and prepackaged assays The kits and assays can include, but are not limited to, tubes (e.g., microcentrifuge tubes), vials, microtiter plates, microfluidic devices, and other high throughput devices. The kits can contain control samples with various predetermined amounts of ATIII or tubes or wells coated with predetermined amounts of ATIII, heparin or tubes or wells coated with heparin, the described aptamers, and optionally thrombin (e.g., alpha thrombin). The kits and assays can further contain instructions for use.

### Example 13. Purification of ATII from a Biological Sample—Heparin Bead Assay

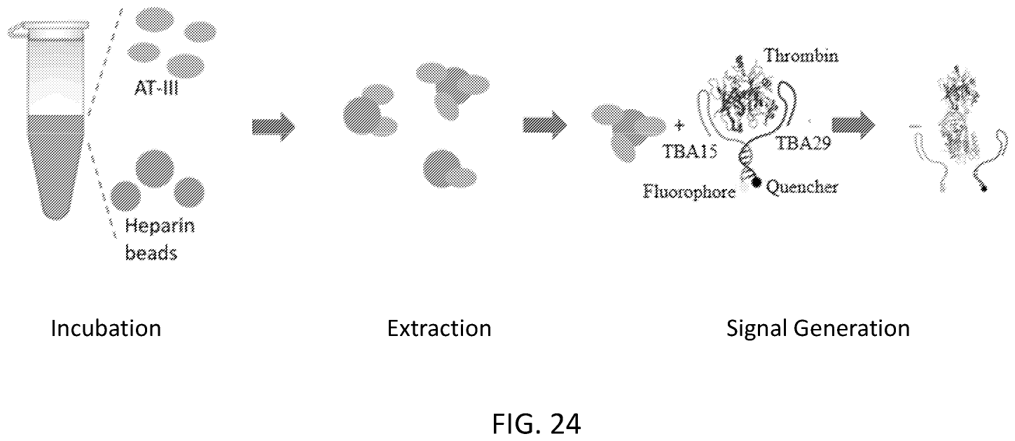

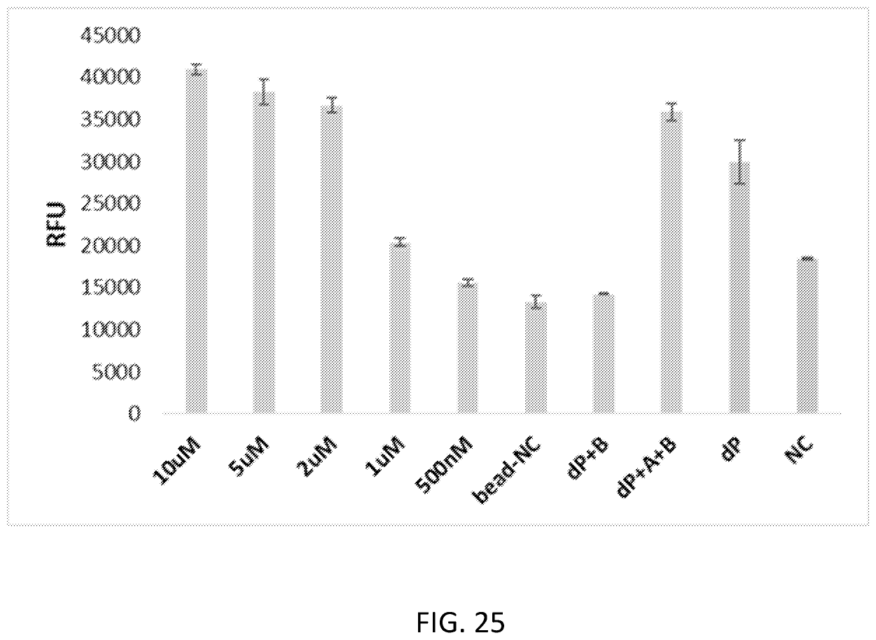

A sample was collected from a subject and incubated with heparin linked to a solid support. In some methods, the sample was then washed to remove components of the sample that were not bound to the heparin. The ATIII assay was then run in the presence of the solid support. FIG. 24 is a schematic depiction of heparin bound sepharose beads extracting ATIII, where remaining proteins are removed and the aptamer-thrombin assay is added to generate a fluorescence signal indicative of the ATIII originally present in the plasma sample.

The following samples were analyzed as described above:
(a) 10 μM: TBA15-F9+TBA29-D9+Thrombin complexes+heparin beads+10 μM ATIII
(b) 5 μM: TBA15-F9+TBA29-D9+Thrombin complexes+heparin beads+5 μM ATIII
(c) 2 μM: TBA15-F9+TBA29-D9+Thrombin complexes+heparin beads+2 μM ATIII
(d) 1 μM: TBA15-F9+TBA29-D9+Thrombin complexes+heparin beads+1 μM ATIII
(e) 500 nM: TBA15-F9+TBA29-D9+Thrombin complexes+heparin beads+500 nM ATIII
(f) bead-NC: TBA15-F9+TBA29-D9+Thrombin complexes+heparin beads+0 nM ATIII
(g) dP+B: TBA15-F9+TBA29-D9+Thrombin complexes+heparin beads+ATIII deficient plasma (5 μL plasma was diluted in 95 μL PBS).
(h) dP+A+B: TBA15-F9+TBA29-D9+Thrombin complexes+heparin beads+ATIII deficient plasma+ATIII (5 μL plasma was diluted in 95 μL 2 μM ATIII).
(i) dP: TBA15-F9+TBA29-D9+Thrombin complexes+heparin+deficient plasma
(j) NC: TBA15-F9+TBA29-D9+Thrombin complexes+heparin.

FIG. 25 shows that ATIII from plasma bound to the heparin is detected in the assay. The signal of dP+B (deficient plasmid plus heparin beads) is similar to negative control beads, indicating that after extraction, the non-specific molecules in the sample that can trigger the ATIII-independent signal were effectively eliminated; compare with signal generated for deficient plasmid (dP) that was not purified with heparin beads. dP+A+B simulated plasma sample from a subject, and generated a high signal. Adding ATIII to the sample (dP+A+B) resulted in high detectable signal. Thus, use of heparin beads increased specificity of the assay in detecting ATIII in plasma.

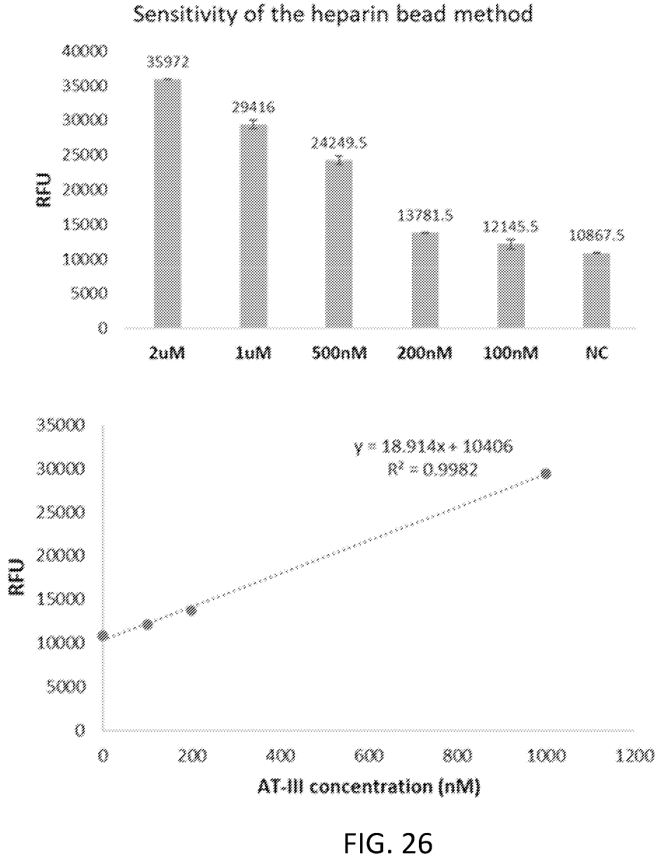

FIG. 26 shows that the described method can detect as little at 100 nM ATIII, which has higher sensitivity compared to the ATIII assay run in the absence of heparin (1 μM detection limit) or the ATIII assay run in the presence of added heparin but without purification of the sample (500 nM limit of detection).

### Example 14. Buffer Analysis

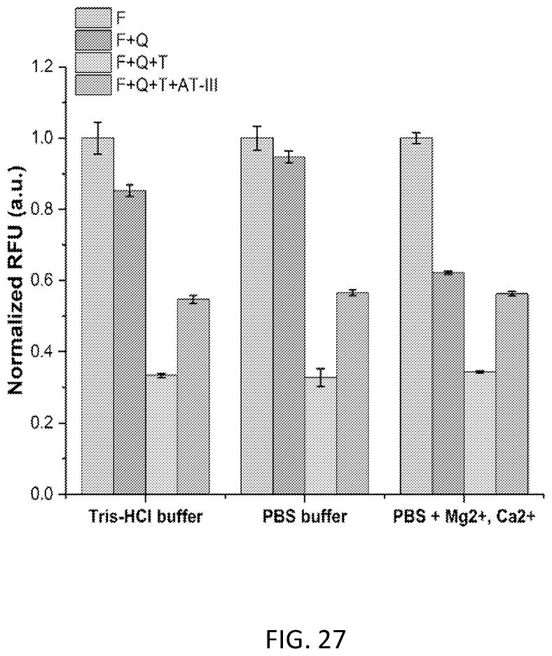

The performance of the assay was tested in different buffers including the tris-HCl buffer, PBS buffer and PBS buffer with Mg²⁺ and Ca²⁺. Intensities were normalized to 200 nM TBA15-F9. In comparison with Tris HCl and PBS buffer with Mg²⁺ and Ca²⁺, PBS buffer provided higher signal in TBA15-F9+TBA29-D9 and TBA15-F9+TBA29-D9+Thrombin+ATIII. Self-hybridization of TBA15-F9+TBA29-D9 in PBS without Mg2+ and Ca2+ was weak, and TBA15-F9+TBA29-D9+Thrombin complex was stable (FIG. 27).

### Example 15. Formation Kinetics

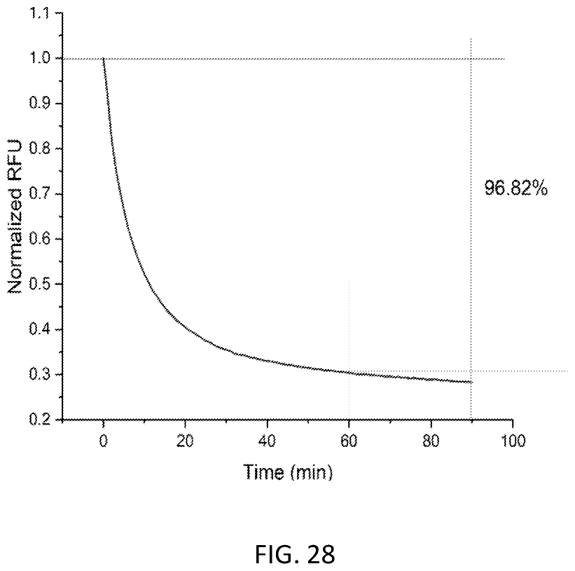

Kinetics of formation of TBA15-F9+TBA29-D9+Thrombin complexes was analyzed. As shown in FIG. 28, 96.82% of stable TBA15-F9+TBA29-D9+Thrombin complexes were formed in 60 minutes compared to 90 minutes. 60 minutes is therefore adequate time for formation of TBA15-F9+TBA29-D9+Thrombin complexes for use in the assay.

### Example 16. Limit of Detection Analysis

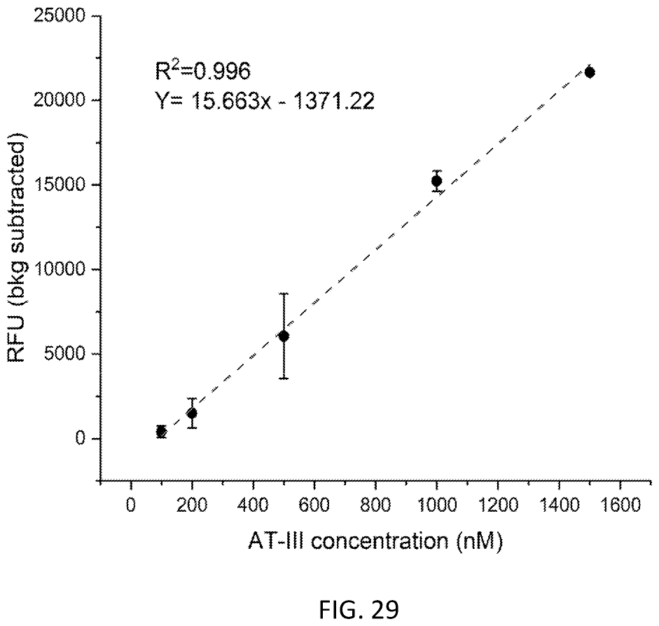

The LoD of the described assay was calculated to be 55.16 nM using 3 Standard deviations with an assay time of 30 min. The results shown in FIG. 29 show that the assay is sensitive enough to detect the ATIII at levels below the level of ATIII normally present in plasma, which is about 2 μM.

1. A system for determining a level of anti-thrombin (ATIII) in a subject comprising:
a column defining an inlet and an outlet, wherein heparin beads are retained within the column;
a manifold connected to the inlet of the column, wherein the manifold comprises a plurality of ports, each port configured to receive a fluid, wherein the manifold conducts the fluid from each port to the inlet of the column;
a first syringe comprising a first washing buffer received at a first port of the plurality of ports of the manifold;
a second syringe comprising plasma received at a second port of the plurality of ports of the manifold;
a third syringe comprising a second washing buffer received at a third port of the plurality of ports of the manifold; and
a fourth syringe comprising an aptamer-thrombin complex received at a fourth port of the plurality of ports of the manifold, wherein the aptamer-thrombin complex comprises a first signaling aptamer comprising a first thrombin-specific aptamer, a first hybridization sequence, and a fluorescent label, a second signaling aptamer comprising a second thrombin-specific aptamer, a second hybridization sequence, and a quencher and thrombin, wherein the first and second hybridization sequences are complementary to each other, and wherein fluorescence of the fluorescent label is quenched when the first and second thrombin-specific signaling aptamers are bound to the thrombin.

2. The system of claim 1, further comprising a substrate, wherein the substrate comprises a waste reservoir separated from the column by a waste valve and a fluorescence reservoir separated from the column by a fluorescence valve.

3. The system of claim 1, wherein the heparin beads retained within the column are washed in response to the first syringe driving the first washing buffer through the inlet of the column.

4. The system of claim 3, wherein the heparin beads are incubated with the plasma in response to the second syringe driving the plasma through the inlet of the column, wherein the heparin beads capture thereon ATIII during incubation with the plasma.

5. The system of claim 4, wherein the heparin beads with the captured ATIII are washed by the second washing buffer in response to the third syringe driving the second washing buffer through the inlet of the column.

6. The system of claim 5, wherein the heparin beads with the captured ATIII are incubated with the aptamer-thrombin complex in response to the fourth syringe driving the aptamer-thrombin complex through the inlet of the column, wherein the ATIII captured on the heparin beads trigger release of aptamer-F to a solution in the column for signal generation.

7. The system of claim 6, wherein fluorescence of the solution is read to determine a level of ATIII in the plasma.

8. A method for determining a level of anti-thrombin (ATIII) in a subject comprising:
driving a first washing buffer through a column within which are suspended heparin beads;
driving a sample through the column, wherein the sample comprises a plasma from the subject;
driving a second washing buffer through the column;
driving a solution comprising an aptamer-thrombin complex through the column, wherein the aptamer-thrombin complex comprises a first signaling aptamer comprising a first thrombin-specific aptamer, a first hybridization sequence, and a fluorescent label, a second signaling aptamer comprising a second thrombin-specific aptamer, a second hybridization sequence, and a quencher and thrombin, wherein the first and second hybridization sequences are complementary to each other, and wherein fluorescence of the fluorescent label is quenched when the first and second thrombin-specific signaling aptamers are bound to the thrombin; and
collecting an eluent from the column, wherein the eluent comprises the aptamer-thrombin complex and ATIII from the sample
measuring fluorescence in the solution, wherein the level of fluorescence is indicative of a level of ATIII in the plasma.

9. The method of claim 8, wherein the first washing buffer is driven through the column by a first syringe, wherein the plasma is driven through the column by a second syringe, wherein the second washing buffer is driven through the column by a third syringe, and wherein the aptamer-thrombin complex is driven through the column by a fourth syringe.

10. The method of claim 8, wherein driving the first washing buffer through the column within which the heparin beads are suspended washes the heparin beads.

11. The method of claim 10, wherein driving the sample through the column captures ATIII present in the sample on the heparin beads.

12. The method of claim 11, wherein driving the second washing buffer through the column purifies the ATIII present in the sample from one or more components present in the sample.

13. The method of claim 12, wherein ATIII present in the sample disrupts binding of the first and second signaling aptamer from the thrombin, resulting in an increase in fluorescence of the fluorescent label.

14. An apparatus for determining a level of anti-thrombin (ATIII) in a subject comprising:
a column defining an inlet and an outlet;
a manifold defining a plurality of ports and a manifold outlet, wherein the manifold outlet is connected to the inlet of the column;
a substrate defining a waste flow path extending between a substrate inlet and a waste reservoir, and a fluorescence flow path extending between the substrate inlet and a fluorescence reservoir;
a waste valve configured to open and close the waste flow path between the substrate inlet and the waste reservoir; and
a fluorescence valve configured to open and close the fluorescence flow path between the substrate inlet and the fluorescence reservoir.

15. The apparatus of claim 14, further comprising:
a first syringe received at a first port of the plurality of ports of the manifold;
a second syringe received at a second port of the plurality of ports of the manifold;
a third syringe received at a third port of the plurality of ports of the manifold; and
a fourth syringe received at a fourth port of the plurality of ports of the manifold.

16. The apparatus of claim 15, wherein the first syringe holds a first washing buffer, the second syringe holds plasma, the third syringe holds a second washing buffer, and the fourth syringe holds an aptamer-thrombin complex, wherein the aptamer-thrombin complex comprises a first signaling aptamer comprising a first thrombin-specific aptamer, a first hybridization sequence, and a fluorescent label, a second signaling aptamer comprising a second thrombin-specific aptamer, a second hybridization sequence, and a quencher and thrombin, wherein the first and second hybridization sequences are complementary to each other, and wherein fluorescence of the fluorescent label is quenched when the first and second thrombin-specific signaling aptamers are bound to the thrombin.

17. The apparatus of claim 16, wherein, in response to sequential driving of contents of the first syringe, the second syringe, the third syringe, and the fourth syringe through the column, the fluorescence reservoir fluoresces based on a level of ATIII within the plasma.

18. A kit for determining the level of anti-thrombin (ATIII) in a subject, comprising:
(a) a first signaling aptamer comprising a first thrombin-specific aptamer, a first hybridization sequence, and a fluorescent label;
(b) a second signaling aptamer comprising a second thrombin-specific aptamer, a second hybridization sequence, and a quencher
(c) thrombin; and
(d) heparin beads,
wherein the first and second hybridization sequences are complementary to each other, and wherein fluorescence of the fluorescent label is quenched when the first and second thrombin-specific signaling aptamers are bound to thrombin.

19. The kit of claim 18, wherein the first signaling aptamer, the second signaling aptamer and the thrombin form a complex.

20. The kit of claim 18, wherein the first signaling aptamer, the second signaling aptamer and the thrombin are provided in a ratio of about 11:1:1 to about 1:1:1.25.

21. The kit of claim 18, wherein the first signaling aptamer, the second signaling aptamer and and/or the thrombin are provided in a buffer comprises a tris-HCl buffer, a phosphate buffered (PBS) buffer, or a PBS buffer that does not contain magnesium or calcium.

22. The kit of claim 19, wherein the thrombin, first signaling aptamer, and second signaling aptamer are provided in a complex at a concentration of about 200 nM.

23. A method of determining a level of ATIII in a subject, comprising:
(a) contacting a sample from the subject with heparin linked to a support, wherein any ATIII in the sample binds to the heparin;
(b) purifying the sample to remove one or more components of the sample that do not bind to the heparin to form a purified sample;
(c) contacting the purified sample with an aptamer-thrombin complex to form an assay solution, wherein the aptamer-thrombin complex comprises a first signaling aptamer comprising a first thrombin-specific aptamer, a first hybridization sequence, and a fluorescent label, a second signaling aptamer comprising a second thrombin-specific aptamer, a second hybridization sequence, and a quencher and thrombin, wherein the first and second hybridization sequences are complementary to each other, and wherein fluorescence of the fluorescent label is quenched when the first and second thrombin-specific signaling aptamers are bound to the thrombin; and
(d) measuring fluorescence in the assay solution wherein the level of fluorescence is proportional to the level of ATIII in the sample.

24. The system of any one of claims 1-7, the method of any one of claims 8-13 and 22, the apparatus of claim 16 or 17, or the kit of any one of claims 18-22, wherein the first thrombin-specific aptamer comprises TBA15 and the second thrombin-specific aptamer comprises TBA29.

25. The system, method, apparatus, or kit of claim 24 wherein the first hybridization sequence comprises the sequence 5′-GTCGTAAGT-3′ and the second hybridization sequences comprises the sequence 5′-ACTTACGAC-3′ or the first hybridization sequence comprises the sequence 5′-ACTTACGAC-3′ and the second hybridization sequences comprises the sequence 5′-GTCGTAAGT-3′.

26. The system, method, apparatus, or kit of claim 24 or 25, wherein the first and/or second signaling aptamers comprises a linker, wherein the linker connects the thrombin-specific aptamer to the hybridization sequence.

27. The system, method, apparatus, or kit of claim 26, wherein linker comprises polyethylene glycol (PEG), or PEG₆.
